(19) United States
(12) Patent Application Publication
(10) Pub. No.: US 2006/0188468 A1
(43) Pub. Date: Aug. 24, 2006

(54) Cosmetic and pharmaceutical substances based on polyelectrolyte complexes

(75) Inventors: Son Nguyen-Kim, Hemsbach (DE); Gabi Muller, Ludwigshafen (DE); Claudia Wood, Weinheim (DE); Peter Hossel, Schifferstadt (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 10/564,627

(22) PCT Filed: Jul. 13, 2004
(86) PCT No.: PCT/EP04/07742

§ 371 Date: Jan. 13, 2006

(30) Foreign Application Priority Data
Jul. 14, 2003 (DE) 103 31 870.4

(51) Int. Cl. A61K 8/81

(52) U.S. Cl. 424/70.15

(57) ABSTRACT

The present invention relates to cosmetic and pharmaceutical compositions which comprise at least one polyelectrolyte complex which comprises a copolymer with cationogenic groups based on vinylimidazole and an acid-group-containing polymer. The invention further relates to the use of these polyelectrolyte complexes.

The present invention relates to cosmetic and pharmaceutical compositions which comprise at least one polyelectrolyte complex which comprises a copolymer with cationogenic and/or cationic groups based on vinylimidazole and an acid-group-containing polymer. The invention further relates to the use of these polyelectrolyte complexes.

Polymers with a relatively large number of ionically dissociable groups in the main chain and/or a side chain are referred to as polyelectrolytes. An ionogenic or ionic polymer can react with an oppositely chargeable or charged polymer to form a polyelectrolyte complex (symplex). Polyelectrolytes with a sufficient number of dissociable groups are water-soluble or water-dispersible and have found diverse applications in the field of coatings, paper auxiliaries, in the manufacture of textiles, and specifically in pharmacy and cosmetics.

Cosmetically and pharmaceutically acceptable water-soluble polymers are used widely in cosmetics and medicine. In soaps, creams and lotions, for example, they are usually used as formulation agents, e.g. as thickener, foam stabilizer or water absorbent, or else to alleviate the irritative effect of other ingredients or to improve the dermal application of active ingredients. Their task in hair cosmetics is to influence the properties of the hair. In pharmacy, they are used, for example, as coatings or binders for solid drug forms.

For hair cosmetics, film-forming polymers are used, for example, as conditioners in order to improve the dry and wet combability, the feel to the touch, shine and appearance, and to impart antistatic properties to the hair. It is known to use water-soluble polymers with cationic functionalities in hair conditioners which have a greater affinity to the surface of the hair, which is negatively charged as a consequence of its structure, and prevent electrostatic charging of the hair. The structure and mode of action of various hair treatment polymers are described in Cosmetic & Toiletries 103 (1988) 23. Standard commercial cationic conditioning polymers are e.g. cationic hydroxyethylcellulose, cationic polymers based on N-vinylpyrrolidone, e.g. copolymers of N-vinylpyrrolidone and quaternized N-vinylimidazole or copolymers of acrylamide and diallyldimethylammonium chloride.

To set hairstyles, use is made, for example, of vinyllactam homo- and copolymers and carboxylate-group-containing polymers. Requirements for hair-setting resins are, for example, strong hold at high atmospheric humidity, elasticity, ability to be washed out of the hair, compatibility in the formulation and a pleasant feel of the hair treated therewith.

It is often problematical to provide products with a complex profile of properties. Thus, there is a need for polymers for cosmetic compositions which are able to form essentially smooth, tack-free films which give the hair and the skin good sensorily ascertainable properties, such as a pleasant feel, and at the same time have a good conditioning effect or setting effect. In addition, esthetic requirements are increasingly placed by the consumer on cosmetic and pharmaceutical products. Thus, in the case of products of this type, a preference for clear, opaque formulations is currently being observed. For this purpose, the polymers used must have good compatibility with as many of the other cosmetic formulation constituents as possible.

EP-A-670 333 describes crosslinked water-soluble polymer dispersions which are obtainable by polymerization of a monomer mixture comprising at least one water-soluble monomer, at least one crosslinker, and optionally hydrophobic and/or amphiphilic monomers in the presence of a polymeric dispersant. As well as a large number of others, water-soluble monomers which may be used are also N-vinylpyrrolidone and monomers with cationic/cationizable groups, such as N-vinylimidazole. The polymeric dispersants may be polyelectrolytes which contain, in copolymerized form, for example salts of (meth)acrylic acid as anionic monomer building blocks or quaternized derivatives of N,N-dimethylaminoethyl (meth)acrylate as cationic building blocks. A use of the polymer dispersions in cosmetics is not described.

EP-A-929 285 teaches the use of water-soluble copolymers which contain, in copolymerized form, vinylcarboxamide units and vinylimidazole units as a constituent of cosmetic compositions. Polyelectrolyte complexes of these copolymers with carboxylic-acid-group-containing polymers are not disclosed in this document.

WO 00/27893 describes aqueous polymer dispersions based on N-vinylcarboxamides and optionally further comonomers, where N-vinylpyrrolidone, N-vinylimidazole and N-vinylimidazole derivatives are also mentioned as well as a large number of others. The polymerization takes place in the presence of at least one polymeric dispersant. A use of these polymer dispersions in cosmetics is described only very generally and without demonstration by a working example.

EP-A-1038891 describes water-soluble or water-dispersible polymeric salts of at least one polymer and at least one oppositely charged neutralizing agent.

WO 02/41856 describes the use of polymer dispersions which are obtainable by polymerization of at least one water-soluble monomer in an aqueous salt solution which comprises at least one polyelectrolyte as dispersant for the cosmetic treatment of keratin materials. In addition, the dispersions comprise at least one agent for adjusting the viscosity, for example a polycarboxylic acid or a salt thereof. Water-soluble monomers which may be used are cationic, anionic and nonionic monomers, preference being given to monomer mixtures which comprise at least one cationic monomer, and optionally additionally acrylic acid and/or acrylamide.

The unpublished German patent application 102 61 197.1 describes an aqueous dispersion obtainable by free-radical polymerization of

a) at least one N-vinyl-containing monomer,

b) at least one polymeric dispersant,

c) at least one polymeric precipitation agent,

d) at least one crosslinker,

e) optionally further monomers,

f) optionally at least one regulator,

g) optionally a buffer substance,

where the weight ratio of b) to c) is in the range from 1:50 to 1:0.02, and to the use thereof in cosmetic preparations.

The unpublished German patent application P 102 61 750.3 describes an ampholytic copolymer which is obtainable by free-radical copolymerization of
a) at least one ethylenically unsaturated compound with at least one anionogenic and/or anionic group,
b) at least one ethylenically unsaturated compound with at least one cationogenic and/or cationic group,
c) at least one unsaturated amide-group-containing compound
   and optionally further comonomers. Also described are polyelectrolyte complexes which contain such an ampholytic copolymer, and also cosmetic or pharmaceutical compositions based on these ampholytic copolymers and polyelectrolyte complexes.

There is still a need for improvement in the case of the polymers known from the prior art for cosmetic and pharmaceutical applications. This is true especially for polymers which, as well as having good film-forming properties, also permit adjustment of the rheological properties of the products, meaning that they can be formulated, for example, in the form of mousses or gels. Also desirable are clear aqueous preparations of these polymers which are accordingly characterized by good compatibility with other formulation constituents. Polymers for use in hair cosmetics should also be notable for producing elastic hairstyles coupled with simultaneously strong hold even at high atmospheric humidity, good ability to be washed out and by good feel of the hair treated therewith.

It is an object of the present invention to find a polymer composition which does not have the disadvantages mentioned.

We have found that this object is achieved by polyelectrolyte complexes which comprise a copolymer with cationogenic groups based on vinylimidazole and a carboxylic-acid-group-containing polymer.

The invention therefore provides a cosmetic or pharmaceutical composition comprising

A) at least one polyelectrolyte complex comprising

A1) at least one water-soluble or water-dispersible copolymer with cationogenic groups which comprises, in copolymerized form, vinylimidazole and/or a derivative thereof and at least one further monomer copolymerizable therewith, and
A2) at least one acid-group-containing polymer, and
   B) at least one cosmetically acceptable carrier.

For the purposes of the present invention, the term alkyl includes straight-chain and branched alkyl groups. Suitable short-chain alkyl groups are e.g. straight-chain or branched C₁-C₇-alkyl, preferably C₁-C₆-alkyl and particularly preferably C₁-C₄-alkyl groups. These include, in particular, methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl etc.

Suitable longer-chain C₈-C₃₀-alkyl or C₈-C₃₀-alkenyl groups are straight-chain and branched alkyl or alkenyl groups. Preference is given here to predominantly linear alkyl radicals, as arise also in natural or synthetic fatty acids and fatty alcohols, and oxo alcohols, which may optionally be additionally mono-, di- or polyunsaturated. These include e.g. n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene) etc.

Cycloalkyl is preferably C₅-C₈-cycloalkyl, such as cyclopentyl, cyclohexyl, cycloheptyl or cyclooctyl.

Aryl includes unsubstituted and substituted aryl groups and is preferably phenyl, tolyl, xylyl, mesityl, naphthyl, fluorenyl, anthracenyl, phenanthrenyl, naphthacenyl and in particular is phenyl, tolyl, xylyl or mesityl.

In the text below, compounds which may be derived from acrylic acid and methacrylic acid are sometimes referred to in abbreviated form by adding the syllable “(meth)” to the compound derived from acrylic acid.

The polyelectrolyte complexes according to the invention can advantageously be formulated as gels under standard conditions (20° C.). “Gel-like consistency” is exhibited by formulations which have a higher viscosity than a liquid and which are self-supporting, i.e. they retain a shape given to them without shape-stabilizing covering. In contrast to solid formulations, however, gel-like formulations can be readily deformed under the application of shear forces. The viscosity of the gel-like compositions is preferably in a range from greater than 600 to about 60 000 mPas, particularly preferably from 6 000 to 30 000 mPas. The gels are preferably hair gels.

For the purposes of the present invention, water-soluble monomers and polymers are understood as meaning monomers and polymers which dissolve in water in an amount of at least 1 g/l at 20° C. Water-dispersible monomers and polymers are understood as meaning monomers and polymers which disintegrate into dispersible particles under the application of shear forces, for example by stirring. Hydrophilic monomers are preferably water-soluble or at least water-dispersible. The polyelectrolyte complexes according to the invention are generally water-soluble.

To prepare the copolymers A1), preference is given to using monomers with initially uncharged, i.e. with cationogenic, groups. This is true especially for vinylimidazole and/or derivatives thereof. Thus, according to the invention, the copolymer A1) comprises, in copolymerized form, vinylimidazole and/or a derivative thereof in at least partially uncharged form (i.e. a nonprotonated and nonquaternized form). A preferred embodiment relates to compositions which comprise, in copolymerized form, N-vinylimidazole and derivatives thereof in completely uncharged form. Compared with those based on essentially quaternized or protonated vinylimidazole, such compositions generally exhibit an improvement in at least one of the following properties: they exhibit better conditioning properties, are clearer, less tacky and/or of higher viscosity.

It is, however, also possible to use further charged N-vinylimidazole (derivatives) different from uncharged N-vinylimidazole (derivatives) for the preparation of the copolymers A1). Furthermore, it is possible to use further cationogenic and/or cationic monomers (i.e. further monomers in non-, partially or completely protonated and/or quaternized form) which are different from N-vinylimidazole (derivatives).

The copolymers A1) preferably comprise no monomers with anionogenic and/or anionic groups in copolymerized form.

The carboxylic-acid-group-containing copolymers A2) are polyelectrolytes with a relatively large number of anionically dissociable groups in the main chain and/or a side chain. They are capable of forming polyelectrolyte complexes (symplexes) with the copolymers A1).

In a preferred embodiment, the polyelectrolyte complexes used in the compositions according to the invention have an excess of anionogenic/anionic groups.

The pH of a 0.1 molar aqueous solution of the polyelectrolyte complexes used in the compositions according to the invention is, at a temperature of 20° C., preferably in the range from 5.5 to 9.0, particularly preferably from 5.8 to 8.5 and in particular from 6.0 to 8.3. Since the polyelectrolyte complexes used according to the invention generally act as buffers, the pH values of their aqueous solutions are generally relatively stable toward dilution and the addition of acid or base within a wide range.

To adjust the pH of aqueous solutions of the polyelectrolyte complexes, all inorganic or organic acids and bases are in principle suitable, in particular those which are water-soluble. Suitable acids are, for example, carboxylic acids, such as lactic acid, citric acid or tartaric acid or mineral acids, such as phosphoric acid, sulfuric acid or hydrochloric acid. Suitable bases are, for example, alkali metal and alkaline earth metal hydroxides, ammonia, and primary, secondary and tertiary amines, such as triethylamine, and amino alcohols, such as triethanolamine, methyldiethanolamine, dimethylethanolamine and 2-amino-2-methylpropanol. Suitable buffers are preferably salts of the abovementioned weak acids, preferably alkali metal and alkaline earth metal salts, such as sodium, potassium, ammonium or magnesium salts. Preferred buffer substances are sodium acetate, sodium citrate, sodium pyrophosphate, potassium pyrophosphate, sodium dihydrogenphosphate, disodium hydrogenphosphate, sodium hydrogencarbonate and/or sodium borate. Said pH-adjusting substances may be used either individually or else in mixtures. The buffer substances may be added to adjust the pH together or else in each case individually.

Monomer a)

The copolymer A1) used in the compositions according to the invention preferably comprises, in copolymerized form, 1 to 70% by weight, particularly preferably 5 to 60% by weight, in particular 7 to 55% by weight, based on the total weight of the monomers used for the polymerization, of at least one monomer a). In a particular embodiment, the proportion of monomers a) is at most 25% by weight.

The copolymer A1) preferably comprises, as monomer a), at least one N-vinylimidazole compound of the formula (III) 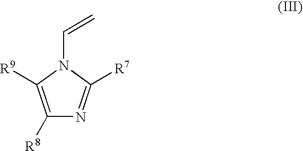
in copolymerized form, in which R⁷ to R⁹, independently of one another, are hydrogen, C₁-C₄-alkyl or phenyl.

Examples of compounds of the formula (III) are given in table 1 below:

TABLE 1

| R⁷ | R⁸ | R⁹ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

A preferred monomer a) is 1-vinylimidazole (N-vinylimidazole).

Monomer b)

The copolymer A1) used in the compositions according to the invention preferably comprises, in copolymerized form, 30 to 99% by weight, particularly preferably 40 to 95% by weight, in particular 45 to 93% by weight, based on the total weight of the monomers used for the polymerization, of at least one further monomer b) which is copolymerizable therewith. In a particular embodiment, the proportion of monomers b) is at least 75% by weight.

The copolymer A1) preferably comprises, in copolymerized form, additionally at least one N-vinyllactam b). Suitable monomers b) are unsubstituted N-vinyllactams and N-vinyllactam derivatives which may, for example, have one or more C₁-C₆-alkyl substituents, such as methyl, ethyl, n-propyl, isopropyl, n-butyl, sec-butyl, tert-butyl etc. These include, for example, N-vinylpyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc. Preference is given to using N-vinylpyrrolidone and N-vinylcaprolactam.

In a particular embodiment, the compositions according to the invention comprise, as component A1), a copolymer which consists only of monomer units of the abovementioned monomers a) and b). These copolymers A1) then preferably comprise, in copolymerized form, 0.5 to 40% by weight, particularly preferably 1 to 30% by weight, in particular 2 to 20% by weight, of at least one monomer a). Correspondingly, these copolymers A1) comprise, in copolymerized form, preferably 60 to 99.5% by weight, particularly preferably 70 to 99% by weight, in particular 80 to 98% by weight, of at least one monomer b).

In a further suitable embodiment, the compositions according to the invention comprise, as component A1), a copolymer which, in addition to the abovementioned monomers a) and b), comprises at least one further, different monomer in copolymerized form.

Monomer c)

The copolymers A1) can additionally comprise, in copolymerized form, at least one nonionic water-soluble monomer c) which is different from the components a) and b) and copolymerizable therewith.

The proportion of monomer c) is preferably 0 to 50% by weight, particularly preferably 5 to 40% by weight, in particular 10 to 35% by weight, based on the total weight of the monomers used for the polymerization.

Component c) is preferably chosen from N-vinylamides of saturated C₁-C₈-monocarboxylic acids, primary amides of α,β-ethylenically unsaturated monocarboxylic acids and N-alkyl and N,N-dialkyl derivatives thereof which, in addition to the carbonyl carbon atom of the amide group, have at most 8 further carbon atoms, esters of α,β-ethylenically unsaturated mono- and dicarboxylic acids with diols, amides of α,β-ethylenically unsaturated mono- and dicarboxylic acids with amino alcohols which have a primary or secondary amino group, polyether acrylates and mixtures thereof.

Open-chain N-vinylamide compounds suitable as monomers c) are, for example, N-vinylformamide, N-vinyl-N-methylformamide, N-vinylacetamide, N-vinyl-N-methylacetamide, N-vinyl-N-ethylacetamide, N-vinylpropionamide, N-vinyl-N-methylpropionamide and N-vinylbutyramide.

Suitable additional monomers c) are also acrylamide, methacrylamide, N-methyl(meth)acrylamide, N-ethyl(meth)acrylamide, N-propyl(meth)acrylamide, N-(n-butyl)(meth)acrylamide, N-(tert-butyl)(meth)acrylamide, N,N-dimethyl(meth)acrylamide, N,N-diethyl(meth)acrylamide, piperidinyl(meth)acrylamide and morpholinyl(meth)acrylamide.

Suitable additional monomers c) are also 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate and 3-hydroxy-2-ethylhexyl methacrylate.

Suitable additional monomers c) are also 2-hydroxyethylacrylamide, 2-hydroxyethylmethacrylamide, 2-hydroxyethylethacrylamide, 2-hydroxypropylacrylamide, 2-hydroxypropylmethacrylamide, 3-hydroxypropylacrylamide, 3-hydroxypropylmethacrylamide, 3-hydroxybutylacrylamide, 3-hydroxybutylmethacrylamide, 4-hydroxybutylacrylamide, 4-hydroxybutylmethacrylamide, 6-hydroxyhexylacrylamide, 6-hydroxyhexylmethacrylamide, 3-hydroxy-2-ethylhexylacrylamide and 3-hydroxy-2-ethylhexylmethacrylamide.

Suitable monomers c) are also polyether acrylates which, for the purposes of this invention, are understood as meaning generally esters of α,β-ethylenically unsaturated mono- and dicarboxylic acids with polyetherols. Suitable polyetherols are linear or branched substances having terminal hydroxyl groups and containing ether bonds. In general, they have a molecular weight in the range from about 150 to 20 000. Suitable polyetherols are polyalkylene glycols, such as polyethylene glycols, polypropylene glycols, polytetrahydrofurans and alkylene oxide copolymers. Suitable alkylene oxides for the preparation of alkylene oxide copolymers are, for example, ethylene oxide, propylene oxide, epichlorohydrin, 1,2- and 2,3-butylene oxide. The alkylene oxide copolymers can contain the alkylene oxide units in random distribution or in the form of blocks. Preference is given to ethylene oxide/propylene oxide copolymers.

Preferred as components c) are polyether acrylates of the formula II 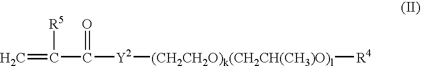
in which
the order of the alkylene oxide units is arbitrary,
k and l, independently of one another, are an integer from 0 to 1000, where the sum of k and l is at least 5,
R⁴ is a hydrogen, C₁-C₃₀-alkyl or C₅-C₈-cycloalkyl,
R⁵ is hydrogen or C₁-C₈-alkyl,
Y² is O or NR⁶, where R⁶ is hydrogen, C₁-C₃₀-alkyl or C₈-C₈-cycloalkyl.

k is preferably an integer from 1 to 500, in particular 3 to 250. l is preferably an integer from 0 to 100.

Preferably R⁵ is hydrogen, methyl, ethyl, n-propyl, isopropyl, n-butyl, sec-butyl, tert-butyl, n-pentyl or n-hexyl, in particular hydrogen, methyl or ethyl.

R⁴ in the formula II is preferably hydrogen, methyl, ethyl, n-propyl, isopropyl, n-butyl, sec-butyl, n-pentyl, n-hexyl, octyl, 2-ethylhexyl, decyl, lauryl, palmityl or stearyl.

Y² in the formula II is preferably O or NH.

Suitable polyether acrylates c) are, for example, the polycondensation products of the abovementioned α,β-ethylenically unsaturated mono- and/or dicarboxylic acids and the acid chlorides, amides and anhydrides thereof with polyetherols. Suitable polyetherols can be prepared easily by reacting ethylene oxide, 1,2-propylene oxide and/or epichlorohydrin with a starter molecule, such as water or a short-chain alcohol R⁴—OH. The alkylene oxides may be used individually, alternately one after the other or as a mixture. The polyether acrylates c) can be used on their own or in mixtures for the preparation of the polymers used according to the invention.

Suitable polyether acrylates are also urethane (meth)acrylates with alkylene oxide groups. Such compounds are described in DE 198 38 851 (component e2)), which is hereby incorporated in its entirety by reference.

The abovementioned monomers c) can in each case be used individually or in the form of arbitrary mixtures.

Monomer d)

The copolymers A1) can additionally comprise, in copolymerized form, at least one water-soluble monomer d) which is chosen from α,β-ethylenically unsaturated water-soluble compounds with cationogenic and/or cationic hydrophilic groups.

The proportion of monomers d) is preferably 0 to 30% by weight, particularly preferably 0.1 to 20% by weight, in particular 0.5 to 15% by weight, based on the total weight of the monomers used for the polymerization.

The cationogenic and/or cationic groups of component d) are preferably nitrogen-containing groups, such as primary, secondary and tertiary amino groups, and quaternary ammonium groups. The nitrogen-containing groups are preferably tertiary amino groups or quaternary ammonium groups. Charged cationic groups can be produced from the amine nitrogens either by protonation or by quaternization, e.g. with the acids and alkylating agents specified above for component a). These include, for example, carboxylic acids, such as lactic acid, or mineral acids, such as phosphoric acid, sulfuric acid and hydrochloric acid, or as alkylating agents C₁-C₄-alkyl halides or sulfates, such as ethyl chloride, ethyl bromide, methyl chloride, methyl bromide, dimethyl sulfate and diethyl sulfate. A protonation or quaternization can generally take place either before or after the polymerization.

Suitable monomers d) are compounds obtainable by protonation or quaternization of component a). Examples of such charged monomers d) are quaternized vinylimidazoles, in particular 3-methyl-1-vinylimidazolium chloride and methosulfate.

Suitable compounds d) are also the esters of α,β-ethylenically unsaturated mono- and dicarboxylic acids with amino alcohols. Preferred amino alcohols are C₂-C₁₂-amino alcohols which are C₁-C₈-dialkylated on the amine nitrogen. Examples of suitable acid components of these esters are acrylic acid, methacrylic acid, fumaric acid, maleic acid, itaconic acid, crotonic acid, maleic anhydride, monobutyl maleate and mixtures thereof. Preference is given to using acrylic acid, methacrylic acid and mixtures thereof as acid component.

Preferred monomers d) are N,N-dimethylaminomethyl (meth)acrylate, N,N-dimethylaminoethyl (meth)acrylate, N,N-diethylaminoethyl (meth)acrylate, N,N-dimethylaminopropyl (meth)acrylate, N,N-diethylaminopropyl (meth)acrylate and N,N-dimethylaminocyclohexyl (meth)acrylate.

Suitable monomers d) are also the amides of the abovementioned α,β-ethylenically unsaturated mono- and dicarboxylic acids with diamines which have at least one primary or secondary amino group. Preference is given to diamines which have one tertiary and one primary or secondary amino group.

Suitable as monomers d) are, for example, N-[2-(dimethylamino)ethyl]acrylamide, N-[2-(dimethylamino)ethyl]methacrylamide, N-[3-(dimethylamino)propyl]acrylamide, N-[3-(dimethylamino)propyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexyl]acrylamide and N-[4-(dimethylamino)cyclohexyl]methacrylamide.

Suitable monomers d) are also N,N-diallylamines and N,N-diallyl-N-alkylamines and acid addition salts and quaternization products thereof. Alkyl here is preferably C₁-C₂₄-alkyl. Preference is given to N,N-diallyl-N-methylamine and N,N-diallyl-N,N-dimethylammonium compounds, such as, for example, the chlorides and bromides. These include, in particular, N,N-diallyl-N,N-dimethylammonium chloride (DADMAC).

Suitable monomers d) are also vinyl- and allyl-substituted nitrogen heterocycles different from component a), such as 2- and 4-vinylpyridine, 2- and 4-allylpyridine, and the salts thereof.

The abovementioned monomers d) can in each case be used individually or in the form of arbitrary mixtures.

Monomer e)

The copolymers A1) can additionally comprise, in copolymerized form, at least one further monomer e). The additional monomers e) are preferably chosen from esters of α,β-ethylenically unsaturated mono- and dicarboxylic acids with C₁-C₃₀-alkanols, N-alkyl- and N,N-dialkylamides of α,β-ethylenically unsaturated monocarboxylic acids which, in addition to the carbonyl carbon atom of the amide group, have at least 9 further carbon atoms, esters of vinyl alcohol and allyl alcohol with C₁-C₃₀-monocarboxylic acids, vinyl ethers, vinylaromatics, vinyl halides, vinylidene halides, C₁-C₈-monoolefins, nonaromatic hydrocarbons with at least two conjugated double bonds and mixtures thereof.

The proportion of monomers e) is preferably 0 to 15% by weight, particularly preferably 0.1 to 10% by weight, based on the total weight of the monomers used for the polymerization.

Suitable additional monomers e) are methyl (meth)acrylate, methyl ethacrylate, ethyl (meth)acrylate, ethyl ethacrylate, n-butyl (meth)acrylate, tert-butyl (meth)acrylate, tert-butyl ethacrylate, n-octyl (meth)acrylate, 1,1,3,3-tetramethylbutyl (meth)acrylate, ethylhexyl (meth)acrylate, n-nonyl (meth)acrylate, n-decyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arrachinyl (meth)acrylate, behenyl (meth)acrylate, lignocerenyl (meth)acrylate, cerotinyl (meth)acrylate, melissinyl (meth)acrylate, palmitoleinyl (meth)acrylate, oleyl (meth)acrylate, linolyl (meth)acrylate, linolenyl (meth)acrylate, stearyl (meth)acrylate, lauryl (meth)acrylate and mixtures thereof. Preferred monomers e) are the esters of α,β-ethylenically unsaturated mono- and dicarboxylic acids with C₁-C₄-alkanols.

Suitable additional monomers e) are also N-(n-octyl)(meth)acrylamide, N-(1,1,3,3-tetramethylbutyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)acrylamide, N-arrachinyl(meth)acrylamide, N-behenyl(meth)acrylamide, N-lignocerenyl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide, N-lauryl(meth)acrylamide.

Suitable additional monomers e) are also vinyl acetate, vinyl propionate, vinyl butyrate and mixtures thereof.

Suitable additional monomers e) are also ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

The abovementioned additional monomers e) can be used individually or in the form of arbitrary mixtures.

Crosslinker f)

The copolymers A1) can, if desired, comprise, in copolymerized form, at least one crosslinker, i.e. a compound with two or more than two ethylenically unsaturated, nonconjugated double bonds.

Crosslinkers are preferably used in an amount of from 0.01 to 3% by weight, particularly preferably 0.1 to 2% by weight, based on the total weight of the monomers used for the polymerization.

Suitable crosslinkers f) are, for example, acrylic esters, methacrylic esters, allyl ethers or vinyl ethers of at least dihydric alcohols. The OH groups of the parent alcohols may here be completely or partially etherified or esterified; however, the crosslinkers contain at least two ethylenically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000. Apart from the homopolymers of ethylene oxide or propylene oxide, it is also possible to use block copolymers of ethylene oxide or propylene oxide or copolymers which contain the ethylene oxide and propylene oxide groups in incorporated form. Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. The polyhydric alcohols can of course also be used following reaction with ethylene oxide or propylene oxide, in the form of the corresponding ethoxylates or propoxylates, respectively. The polyhydric alcohols can also firstly be converted into the corresponding glycidyl ethers by reaction with epichlorohydrin.

Further suitable crosslinkers f) are the vinyl esters or the esters of monohydric, unsaturated alcohols with ethylenically unsaturated C₃-C₆-carboxylic acids, for example acrylic acid, methacrylic acid, itaconic acid, maleic acid or fumaric acid. Examples of such alcohols are allyl alchol, 1-buten-3-ol, 5-hexen-1-ol, 1-octen-3-ol, 9-decen-1-ol, dicyclopentenyl alcohol, 10-undecen-1-ol, cinnamol alcohol, citronellol, crotyl alcohol or cis-9-octadecen-1-ol. It is also possible, however, to esterify the monohydric, unsaturated alcohols with polybasic carboxylic acids, for example malonic acid, tartaric acid, trimellitic acid, phthalic acid, terephthalic acid, citric acid or succinic acid.

Further suitable crosslinkers f) are esters of unsaturated carboxylic acids with the above-described polyhydric alcohols, for example of oleic acid, crotonic acid, cinnamic acid or 10-undecenoic acid.

Suitable crosslinkers f) are also straight-chain or branched, linear or cyclic, aliphatic or aromatic hydrocarbons which have at least two double bonds which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Further suitable crosslinkers f) are the acrylamides, methacrylamides and N-allylamines of at least difunctional amines. Such amines are, for example, 1,2-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacrylic acid, itaconic acid, maleic acid, or at least dibasic carboxylic acids, as have been described above.

Also suitable are triallylamine and triallylmonoalkylammonium salts, e.g. triallylmethylammonium chloride or methylsulfate, as crosslinker f).

Also suitable are N-vinyl compounds of urea derivatives, at least difunctional amides, cyanurates and urethanes, for example of urea, ethyleneurea, propyleneurea or tartardiamide, e.g. N,N′-divinylethyleneurea or N,N′-divinylpropyleneurea.

Further suitable crosslinkers f) are divinyldioxane, tetrallylsilane or tetravinylsilane.

It is of course also possible to use mixtures of the abovementioned compounds f). Preference is given to using water-soluble crosslinkers f).

Particularly preferably used crosslinkers f) are, for example, methylenebisacrylamide, triallylamine and triallylalkylammonium salts, divinylimidazole, pentaerythritol triallyl ether, N,N′-divinylethyleneurea, reaction products of polyhydric alcohols with acrylic acid or methacrylic acid, methacrylic esters and acrylic esters of polyalkylene oxides or polyhydric alcohols which have been reacted with ethylene oxide and/or propylene oxide and/or epichlorohydrin.

Very particularly preferred crosslinkers f) are pentaerythritol triallyl ether, methylenebisacrylamide, N,N′-divinylethyleneurea, triallylamine and triallylmonoalkylammonium salts and acrylic esters of glycol, butanediol, trimethylolpropane or glycerol or acrylic esters of glycol, butanediol, trimethylolpropane or glycerol reacted with ethylene oxide and/or epichlorohydrin.

Preference is given to compositions where the copolymer A1) comprises, in copolymerized form,
a) vinylimidazole,
b) N-vinylpyrrolidone,
c) at least one nonionic water-soluble monomer which is chosen from N-vinylamides of saturated C₁-C₈-monocarboxylic acids and primary amides of α,β-ethylenically unsaturated monocarboxylic acids and N-alkyl and N,N-dialkyl derivatives thereofl which, in addition to the carbonyl carbon atom of the amide group, have at most 8 further carbon atoms,
d) at least one monomer which is chosen from acid salts and quaternization products of vinylimidazole, dimethylaminopropylmethacrylamide and the acid salt and quaternization products of dimethylaminopropylmethacrylamide.

Preference is given to compositions where the copolymer A1) comprises, in copolymerized form,
a) 0.5 to 40% by weight of vinylimidazole and/or a derivative thereof,
b) 20 to 99% by weight of at least one N-vinyllactam,
c) 0 to 50% by weight of at least one nonionic water-soluble monomer which is different from, and copolymerizable with, components a) and b), and
d) 0 to 30% by weight of at least one monomer which is chosen from α,β-ethylenically unsaturated water-soluble compounds with cationogenic and/or cationic hydrophilic groups.

Further preference is given to compositions where the copolymer A1) comprises, in copolymerized form,
a) 1 to 20% by weight of vinylimidazole and/or a derivative thereof,
b) 20 to 80% by weight of at least one N-vinyllactam,
c) 5 to 50% by weight of at least one nonionic water-soluble monomer which is different from, and copolymerizable with, components a) and b), and
d) 0 to 30% by weight of at least one monomer which is chosen from α,β-ethylenically unsaturated water-soluble compounds with cationogenic and/or cationic hydrophilic groups.

Further preference is given to compositions where the copolymer A1) comprises, in copolymerized form,
a) 1 to 10% by weight of vinylimidazole and/or a derivative thereof,
b) 30 to 70% by weight of at least one N-vinyllactam,
c) 10 to 40% by weight of at least one nonionic water-soluble monomer which is different from, and copolymerizable with, components a) and b), and
d) 1 to 20% by weight of at least one monomer which is chosen from α,β-ethylenically unsaturated water-soluble compounds with cationogenic and/or cationic hydrophilic groups.

In a preferred embodiment, the copolymer A1) consists only of repeat units which are derived from the abovementioned monomers a), b) and optionally c) and/or d).

These copolymers A1) preferably consist of repeat units of:
a) 0.5 to 40% by weight, preferably 1 to 35% by weight, of vinylimidazole and/or a derivative thereof,
b) 20 to 99% by weight, preferably 20 to 80% by weight, of at least one N-vinyllactam,
c) 0 to 50% by weight, preferably 5 to 45% by weight, of at least one nonionic water-soluble monomer which is different from, and copolymerizable with, components a) and b), and
d) 0 to 30% by weight, preferably 1 to 20% by weight, of at least one monomer which is chosen from α,β-ethylenically unsaturated water-soluble compounds with cationogenic and/or cationic hydrophilic groups.

The copolymers A1) are prepared in accordance with customary processes known to the person skilled in the art, e.g. by solution, predpitation, suspension or emulsion polymerization. Preference is given to preparation by solution or precipitation polymerization.

Preferred solvents for the solution polymerization are aqueous solvents, such as water and mixtures of water with miscible solvents, for example alcohols, such as methanol, ethanol, n-propanol, isopropanol, n-butanol, sec-butanol, tert-butanol, n-hexanol and cyclohexanol, and glycols, such as ethylene glycol, propylene glycol and butylene glycol, and the methyl or ethyl ethers of dihydric alcohols, diethylene glycol, triethylene glycol, polyethylene glycols with number-average molecular weights up to about 3000, glycerol and dioxane. Particular preference is given to the polymerization in water or a water/alcohol mixture, for example in a water/ethanol mixture.

The precipitation polymerization takes place, for example, in an ester, such as ethyl acetate or butyl acetate, as solvent. The resulting polymer particles precipitate out of the reaction solution and can be isolated by customary processes, such as filtration by means of subatmospheric pressure. In the precipitation polymerization, polymers with higher molecular weights are generally obtained than in the solution polymerization.

The polymerization temperatures are preferably in a range from about 30 to 120° C., particularly preferably 40 to 100° C. The polymerization usually takes place under atmospheric pressure, although it can also proceed under reduced or increased pressure. A suitable pressure range is between 1 and 5 bar.

To prepare the polymers A1), the monomers can be polymerized with the help of initiators which form free radicals.

Initiators which can be used for the free-radical polymerization are the peroxo and/or azo compounds customary for this purpose, for example alkali metal or ammonium peroxydisulfates, diacetyl peroxide, dibenzoyl peroxide, succinyl peroxide, di-tert-butyl peroxide, tert-butyl perbenzoate, tert-butyl perpivalate, tert-butyl peroxy-2-ethylhexanoate, tert-butyl permaleate, cumene hydroperoxide, diisopropyl peroxydicarbamate, bis(o-toluyl) peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, azobisisobutyronitrile, 2,2′-azobis(2-amidinopropane) hydrochloride (V50 from Wako Pure Chemicals Industries, Ltd.), or 2,2′-azobis(2-methylbutyronitrile). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate, H₂O₂/Cu^{l}.

To adjust the molecular weight, the polymerization can take place in the presence of at least one regulator. Regulators which may be used are the customary compounds known to the person skilled in the art, such as, for example, sulfur compounds, e.g. mercaptoethanol, 2-ethylhexyl thioglycolate, thioglycolic acid and dodecylmercaptan, and tribromochloromethane or other compounds which have a regulating effect on the molecular weight of the resulting polymers. A preferred regulator is cysteine.

To achieve the purest possible polymers with a low residual monomer content, the polymerization (main polymerization) may be followed by an afterpolymerization step. The afterpolymerization can take place in the presence of the same initiator system as or a different initiator system from the main polymerization. The afterpolymerization preferably takes place at least at the same temperature as, preferably at a higher temperature than, the main polymerization. If desired, the reaction mixture may, after the polymerization or between the first and the second polymerization step, be subjected to stripping with steam or to steam distillation.

If an organic solvent is used in the preparation of the polymers, then this can be removed by customary processes known to the person skilled in the art, e.g. by distillation at reduced pressure.

The polymerization preferably takes place at a pH in the range from 6 to 9, particularly preferably from 6.5 to 7.5. The pH is adjusted, as also described at the outset, by adding a suitable acid or by adding a suitable base.

The resulting liquid polymer compositions can be converted to powder form by various drying processes, such as, for example, spray-drying, fluidized spray-drying, roll drying or freeze-drying. Preference is given to using spray-drying. The polymer dry powders obtained in this way can advantageously be converted again to an aqueous solution or dispersion by dissolution or redispersion, respectively, in water. Pulverulent copolymers have the advantage of better storability, easier transportability and generally have a lower propensity for microbial attack.

Polymer A2)

As well as comprising at least one of the abovementioned copolymers A1), the polyelectrolyte complexes A) also comprise at least one acid-group-containing polymer A2).

The polyelectrolyte complexes preferably comprise copolymer(s) A1) and polymer(s) A2) in a quantitative weight ratio of about 50:1 to 1:20, particularly preferably from 20:1 to 1:5.

Suitable carboxylic-acid-group-containing polymers A2) are obtainable, for example, by free-radical polymerization of α,β-ethylenically unsaturated monomers. Monomers a2.1) are used here which contain at least one free-radically polymerizable, α,β-ethylenically unsaturated double bond and at least one anionogenic and/or anionic group per molecule.

Suitable carboxylic-acid-group-containing polymers A2) are also carboxylic-acid-group-containing polyurethanes.

The monomers a2.1) are preferably chosen from monoethylenically unsaturated carboxylic acids, sulfonic acids, phosphonic acids and mixtures thereof.

The monomers a2.1) include monoethylenically unsaturated mono- and dicarboxylic acids having 3 to 25, preferably 3 to 6, carbon atoms, which may also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The monomers a2.1) also include the half-esters of monoethylenically unsaturated dicarboxylic acids having 4 to 10, preferably 4 to 6, carbon atoms, e.g. of maleic acid, such as monomethyl maleate. The monomers a2.1) also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxypropylsulfonic acid, styrenesulfonic acid, 2-acrylamido-2-methylpropanesulfonic acid, vinylphosphonic acid and allylphosophonic acid. The monomers a2.1) also include the salts of the abovementioned acids, in particular the sodium, potassium and ammonium salts, and the salts with the abovementioned amines. The monomers a2.1) can also be used as such or as mixtures with one another. The weight fractions given all refer to the acid form.

The component a2.1) is preferably chosen from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof.

The component a2.1) is particularly preferably chosen from acrylic acid, methacrylic acid and mixtures thereof.

The abovementioned monomers a2.1) can in each case be used individually or in the form of arbitrary mixtures.

Comonomers which are suitable in principle for the preparation of the polymers A2) are the compounds a) to f) specified above as components of copolymer A1) with the proviso that the molar fraction of anionogenic and anionic groups which comprises the polymer A2) in copolymerized form is greater than the molar fraction of cationogenic and cationic groups.

In a preferred embodiment, the polymers A2) comprise at least one monomer in copolymerized form which is chosen from the abovementioned crosslinkers f). Reference is made to suitable and preferred crosslinkers f).

The polymers A2) further preferably comprise at least one monomer a2.2) in copolymerized form, which is chosen from compounds of the formula I) 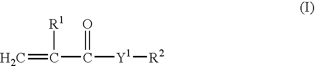
in which
R¹ is hydrogen or C₁-C₈-alkyl,
Y¹ is O, NH or NR³, and
R² and R³, independently of one another, are C₁-C₃₀-alkyl or C₅-C₈-cycloalkyl, where the alkyl groups may be interrupted by up to four nonadjacent heteroatoms or heteroatom-containing groups chosen from O, S and NH,

Preferably, R¹ in the formula I is hydrogen or C₁-C₄-alkyl, in particular hydrogen, methyl or ethyl.

Preferably, R² in the formula I is C₁-C₈-alkyl, preferably methyl, ethyl, n-butyl, isobutyl, tert-butyl or a group of the formula —CH₂—CH₂—NH—C(CH₃)₃.

If R³ is alkyl, then it is preferably C₁-C₄-alkyl, such as methyl, ethyl, n-propyl, n-butyl, isobutyl and tert-butyl.

Suitable monomers a2.2) are methyl (meth)acrylate, methyl ethacrylate, ethyl (meth)acrylate, ethyl ethacrylate, tert-butyl (meth)acrylate, tert-butyl ethacrylate, n-octyl (meth)acrylate, 1,1,3,3-tetramethylbutyl (meth)acrylate, ethylhexyl (meth)acrylate, n-nonyl (meth)acrylat, n-decyl (meth)acrylate, n-undecyl (meth)acrylate, tridecyl (meth)acrylate, myristyl (meth)acrylate, pentadecyl (meth)acrylate, palmityl (meth)acrylate, heptadecyl (meth)acrylate, nonadecyl (meth)acrylate, arrachinyl (meth)acrylate, behenyl (meth)acrylate, lignoceryl (meth)acrylate, cerotinyl (meth)acrylate, melissinyl (meth)acrylate, palmitoleinyl (meth)acrylate, oleyl (meth)acrylate, linolyl (meth)acrylate, linolenyl (meth)acrylate, stearyl (meth)acrylate, lauryl (meth)acrylate and mixtures thereof.

Suitable monomers a2.2) are also acrylamide, methacrylmide, N-methyl(meth)acrylamide, N-ethyl(meth)acrylamide, N-propyl(meth)acrylamide, N-(n-butyl)(meth)acrylamide, N-(tert-butyl)(meth)acrylamide, N,N-dimethyl(meth)acrylamide, N,N-diethyl(meth)acrylamide piperidinyl(meth)acrylamide and morpholinyl(meth)acrylamide, N-(n-octyl)(meth)acrylamide, N-(1,1,3,3-tetramethylbutyl)(meth)acrylamide, N-ethylhexyl(meth)acrylamide, N-(n-nonyl)(meth)acrylamide, N-(n-decyl)(meth)acrylamide, N-(n-undecyl)(meth)acrylamide, N-tridecyl(meth)acrylamide, N-myristyl(meth)acrylamide, N-pentadecyl(meth)acrylamide, N-palmityl(meth)acrylamide, N-heptadecyl(meth)acrylamide, N-nonadecyl(meth)acrylamide, N-arrachinyl(meth)acrylamide, N-behenyl(meth)acrylamide, N-lignoceryl(meth)acrylamide, N-cerotinyl(meth)acrylamide, N-melissinyl(meth)acrylamide, N-palmitoleinyl(meth)acrylamide, N-oleyl(meth)acrylamide, N-linolyl(meth)acrylamide, N-linolenyl(meth)acrylamide, N-stearyl(meth)acrylamide and N-lauryl(meth)acrylamide.

Furthermore, the polymers A2) preferably contain at least one monomer a2.3) in copolymerized form which is chosen from compounds of the formula II 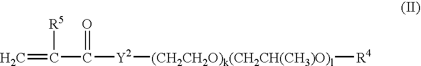
in which
the order of the alkylene oxide units is arbitrary,
k and l, independently of one another, are an integer from 0 to 1000, where the sum of k and l is at least 5,
R⁴ is hydrogen, C₁-C₃₀-alkyl or C₅-C₈-cycloalkyl,
R⁵ is hydrogen or C₁-C₈-alkyl,
Y² is O or NR⁶, where R⁶ is hydrogen, C₁-C₃₀-alkyl or C₅-C₈-cycloalkyl.

Preferably, in the formula II, k is an integer from 1 to 500, in particular 3 to 250. l is preferably an integer from 0 to 100.

Preferably, R⁵ is hydrogen, methyl, ethyl, n-propyl, isopropyl, n-butyl, sec-butyl, tert-butyl, n-pentyl or n-hexyl, in particular hydrogen, methyl or ethyl.

Preferably R⁴ in the formula II is hydrogen, methyl, ethyl, n-propyl, isopropyl, n-butyl, sec-butyl, n-pentyl, n-hexyl, octyl, 2-ethylhexyl, decyl, lauryl, palmityl or stearyl.

Preferably, Y² in the formula II is O or NH.

Suitable polyether acrylates II) are, for example, the polycondensation products of the abovementioned α,β-ethylenically unsaturated mono- and/or dicarboxylic acids and the acid chlorides, amides and anhydrides thereof with polyetherols. Suitable polyetherols can be prepared easily by reacting ethylene oxide, 1,2-propylene oxide and/or epichlorohydrin with a starter molecule, such as water or a short-chain alcohol R⁴—OH. The alkylene oxides can be used individually, alternately one after the other or as a mixture. The polyether acrylates c) can be used on their own or in mixtures for the preparation of the polymers used according to the invention. Suitable polyether acrylates II) are also urethane (meth)acrylates with alkylene oxide groups. Such compounds are described in DE 198 38 851 (component e2)), which is hereby incorporated in its entirety by reference.

Anionic polymers preferred as polymers A2) are, for example, homopolymers and copolymers of acrylic acid and methacrylic acid and salts thereof. These also include crosslinked polymers of acrylic acid, as are available under the INCI name Carbomer. Such crosslinked homopolymers of acrylic acid are available commercially, for example, under the name Carbopol® from BF GOODRICH. Preference is also given to hydrophobically modified crosslinked polyacrylate polymers, such as Carbopol® Ultrez 21 from Noveon.

Polyelectrolyte complexes A) based on homopolymers and copolymers of acrylic acid and methacrylic acid are suitable in an advantageous manner for formulation as gels, for example as setting gels, and also for the formulation of foams.

Further examples of suitable anionic polymers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers which are available under the name Aculyn® from Rohm und Haas. Particularly suitable polymers are also copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid and optionally further vinyl esters (e.g. Luviset® grades), maleic anhydride copolymers, optionally reacted with alcohol, anionic polysiloxanes, e.g. carboxy-functional, t-butyl acrylate, methacrylic acid (e.g. Luviskol® VBM), copolymers of acrylic acid and methacrylic acid with hydrophobic monomers, such as, for example, C₄-C₃₀-alkyl esters of meth(acrylic acid), C₄-C₃₀-alkyl vinyl esters, C₄-C₃₀-alkyl vinyl ether and hyaluronic acid. Examples of anionic polymers are also vinyl acetate/crotonic acid copolymers, as are available commercially, for example, under the names Resyn® (National Starch) and Gafset® (GAF) and vinylpyrrolidone/vinyl acrylate copolymers obtainable, for example, under the trade name Luviflex® (BASF). Further suitable polymers are the vinylpyrrolidone/acrylate terpolymer available under the name Luviflex® VBM-35 (BASF) and sodium sulfonate-containing polyamides or sodium sulfonate-containing polyesters.

The group of suitable anionic polymers also includes, for example, Balance® CR (National Starch; acrylate copolymer), Balance® 0/55 (National Starch; acrylates copolymer), Balance® 47 (National Starch; octylacrylamide/acrylates/butylaminoethyl methacrylate copolymer), Aquaflex® FX 64 (ISP; isobutylene/ethylmaleimide/hydroxyl-ethylmaleimide copolymer), Aquaflex® SF-40 (ISP/National Starch; VP/vinylcaprolactam/DMAPA acrylate copolymer), Allianz® LT-120 (ISP/Rohm & Haas; acrylat/C1-2 succinate/hydroxyacrylate copolymer), Aquarez® HS (Eastman; polyester-1), Diaformer® Z-400 (Clariant; methacryloylethylbetaine/methacrylate copolymer), Diaformer® Z-711 (Clariant; methacryloylethyl N-oxide/methacrylate copolymer), Diaformer® Z-712 (Clariant; methacryloylethyl N-oxide/methacrylate copolymer), Omnirez® 2000 (ISP; monoethyl ester of poly(methyl vinyl ether)/maleic acid in ethanol), Amphomer® HC (National Starch; acrylate/octylacrylamide copolymer), Amphomer® 28-4910 (National Starch; octylacrylamide/acrylate/butyl-aminoethyl methacrylate copolymer), Advantage®D HC 37 (ISP; terpolymer of vinylcaprolactam/vinylpyrrolidone/dimethylaminoethyl methacrylate), Advantage® LC55 and LC80 or LC A and LC E, Advantage® Plus (ISP; VA/butyl maleate/isobornyl acrylate copolymer), Aculyne® 258 (Rohm & Haas; acrylate/hydroxy ester acrylate copolymer), Luviset® P.U.R. (BASF, polyurethane-1), Luviflex® Silk (BASF), Eastman® AQ 48 (Eastman), Styleze® CC-10 (ISP; VP/DMAPA acrylates copolymer), Styleze® 2000 (ISP; VP/acrylates/lauryl methacrylate copolymer), DynamX (National Starch; polyurethane-14 AMP-acrylates copolymer), Resyn XP (National Starch; acrylates/octylacrylamide copolymer), Fixomer A-30 (Ondeo Nalco; polymethacrylic acid (and) acrylamidomethylpropanesulfonic acid), Fixate G-100 (Noveon; AMP-acrylates/allyl methacrylate copolymer).

Suitable copolymers A2) are also the terpolymers of vinylpyrrolidone/C₁-C₁₀-alkyl, cycloalkyl and aryl(meth)acrylates and acrylic acid described in U.S. Pat. No. 3,405,084. Suitable copolymers A2) are also the terpolymers of vinylpyrrolidone, tert-butyl (meth)acrylate and (meth)acrylic acid described in EP-A-0 257 444 and EP-A-0 480 280. Suitable copolymers A2) are also the copolymers described in DE-A42 23 066 which contain at least one (meth)acrylic ester, (meth)acrylic acid, and N-vinylpyrrolidone and/or N-vinylcaprolactam in copolymerized form. Reference is hereby made to the disclosure of these documents.

The preparation of the abovementioned polymers A2) is carried out by known processes, for example of solution, precipitation, suspension or emulsion polymerization, as described above for the copolymers A1).

Suitable carboxylic-acid-group-containing polymers A2) are also carboxylic-acid-group-containing polyurethanes.

EP-A-636361 discloses suitable block copolymers with polysiloxane blocks and polyurethane/polyurea blocks which have carboxylic acid and/or sulfonic acid groups. Suitable silicone-containing polyurethanes are also described in WO 97/25021 and EP-A-751 162.

Suitable polyurethanes are also described in DE-A-42 25 045, which is hereby incorporated in its entirety by reference. These polyurethanes are in principle constructed from
i) at least one compound which contains two or more active hydrogen atoms per molecule,
ii) at least one diol containing carboxylic acid groups, or a salt thereof, and
iii) at least one polyisocyanate.

Component i) is, for example, a diol, diamine, amino alcohol, or mixture thereof. The molecular weight of these compounds is preferably in a range form about 56 to 280. If desired, up to 3 mol % of said compounds can be replaced by triols or triamines.

Diols i) which can be used are, for example, ethylene glycol, propylene glycol, butylene glycol, neopentyl glycol, cyclohexanedimethylol, di-, tri-, tetra-, penta- or hexaethylene glycol and mixtures thereof. Preference is given to using neopentyl glycol and/or cyclohexanedimethylol. Suitable amino alochols i) are, for example, 2-aminoethanol, 2-(N-methylamino)ethanol, 3-aminopropanol, 4-aminobutanol, 1-ethylaminobutan-2-ol, 2-amino-2-methyl-1-propanol, 4-methyl-4-aminopentan-2-ol etc. Suitable diamines i) are, for example, ethylenediamine, propylenediamine, 1,4-diaminobutane, 1,5-diaminopentane and 1,6-diaminohexane, and α,ω-diaminopolyethers which can be prepared by amination of polyalkylene oxides with ammonia.

The component i) may also be a polymer with a number-average molecular weight in the range from about 300 to 5 000, preferably about 400 to 4 000, in particular 500 to 3 000. Polymers i) which can be used, are, for example, polyesterdiols, polyetherols and mixtures thereof. Polyetherols are preferably polyalkylene glycols, e.g. polyethylene glycols, polypropyleneglycols, polytetrahydrofurans etc., block copolymers of ethylene oxide and propylene oxide or block copolymers of ethylene oxide, propylene oxide and butylene oxide which contain, in copolymerized form, the alkylene oxide units in random distribution or in the form of blocks. Suitable polytetrahydrofurans i) can be prepared by cationic polymerization of tetrahydrofuran in the presence of acidic catalysts, such as, for example, sulfuric acid or fluorosulfuric acid. Preparation processes of this type are known to the person skilled in the art. Polyesterdiols i) which can be used preferably have a number-average molecular weight in the range from about 400 to 5 000, preferably 500 to 3 000, in particular 600 to 2 000. Suitable polyesterdiols i) are all those which are usually used for the preparation of polyurethanes, in particular those based on aromatic dicarboxylic acids, such as terephthalic acid, isophthalic acid, phthalic acid, Na or K sulfoisophthalic acid etc., aliphatic dicarboxylic acids, such as adipic acid or succinic acid, etc., and cycloaliphatic dicarboxylic acids, such as 1,2-, 1,3- or 1,4-cyclohexanedicarboxylic acid. Suitable diols are, in particular, aliphatic diols, such as ethylene glycol, propylene glycol, 1,6-hexanediol, neopentyl glycol, diethylene glycol, polyethylene glycols, polypropylene glycols, 1,4-dimethylolcyclohexane, etc.

Suitable compounds ii), which have two active hydrogen atoms and at least one carboxylic acid group per molecule, are, for example, dimethylolpropanoic acid and mixtures which contain dimethylolpropanoic acid.

Component iii) is a customary aliphatic, cycloaliphatic and/or aromatic polyisocyanate, such as tetramethylene diisocyanate, hexamethylene diisocyanate, methylenediphenyl diisocyanate, 2,4- and 2,6-tolylene diisocyanate and isomer mixtures thereof, o- and m-xylylene diisocyanate, 1,5-naphthylene diisocyanate, 1,4-cyclohexylene diisocyanate, dicyclohexylmethane diisocyanate and mixtures thereof, in particular isophorone diisocyanate and/or dicyclohexylmethane diisocyanate. If desired, up to 3 mol % of said compounds may be replaced by triisocyanates.

The polyurethane polymers A2) are prepared by customary processes known to the person skilled in the art. The temperature here is generally in a range from about 40 to 140° C., preferably about 60° to 100° C. The reaction can take place without solvents or in a suitable inert solvent or solvent mixture. Suitable solvents are aprotic-polar solvents, e.g. tetrahydrofuran, ethyl acetate, N-methylpyrrolidone, dimethylformamide and preferably ketones, such as acetone and methyl ethyl ketone. The reaction is preferably carried out under an inert gas atmosphere, such as, for example, under nitrogen.

The acid groups of the polymers A2) may be partially or completed neutralized. At least some of the acid groups are then present in deprotonated form, the counterions preferably being chosen from alkali metal ions, such as Na⁺, K⁺, ammonium ions and organic derivatives thereof etc.

In a preferred embodiment, the compositions according to the invention are in the form of a spray. These compositions preferably comprise at least one polyelectrolyte complex A) in which the carboxylic-acid-group-containing polymer A2) comprises, in copolymerized form,
i) 60 to 90% by weight of at least one compound of the formula I 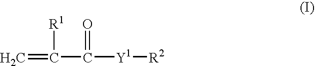
   in which
   R¹ is hydrogen or C₁-C₈-alkyl,
   Y¹ is O, NH or NR³, and
   R² and R³, independently of one another, are C₁-C₃₀-alkyl or C₅-C₈-cycloalkyl, where the alkyl groups may be interrupted by up to four nonadjacent heteroatoms or heteroatom-containing groups which are chosen from O, S and NH,
   ii) 10 to 25% by weight of acrylic acid and/or methacrylic acid,
   iii) 0 to 30% by weight of at least one monomer which is different from, and copolymerizable with, components i) and ii),
   or where the carboxylic-acid-group-containing polymer A2) is a polyurethane.

In a further preferred embodiment, the compositions according to the invention are in the form of a mousse. These compositions preferably comprise at least one polyelectrolyte complex A), in which the carboxylic-acid-group-containing polymer A2) comprises, in copolymerized form,
i) 45 to 85% by weight of at least one compound of the formula I 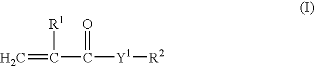
   in which
   R¹ is hydrogen or C₁-C₈-alkyl,
   Y¹ is O, NH or NR³, and
   R² and R³, independently of one another, are C₁-C₃₀-alkyl or C₅-C₈-cycloalkyl, where the alkyl groups may be interrupted by up to four nonadjacent heteroatoms or heteroatom-containing groups chosen from O, S and NH,
ii) 20 to 55% by weight of acrylic acid and/or methacrylic acid,
iii) 0 to 30% by weight of at least one monomer which is different from, and copolymerizable with, components i) and ii).

In a further preferred embodiment, the compositions according to the invention are in the form of a gel. These compositions preferably comprise at least one polyelectrolyte complex A) in which the carboxylic-acid-group-containing polymer A2) comprises, in copolymerized form,
i) 45 to 85% by weight of at least one compound of the formula I 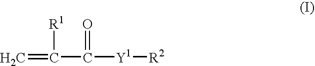
   in which
   R¹ is hydrogen or C₁-C₈-alkyl,
   Y¹ is O, NH or NR³, and
   R² and R³, independently of one another, are C₁-C₃₀-alkyl or C₅-C₈-cycloalkyl, where the alkyl groups may be interrupted by up to four nonadjacent heteroatoms or heteroatom-containing groups which are chosen from O, S and NH,
ii) 20 to 60% by weight of acrylic acid and/or methacrylic acid,
iii) 5 to 50% by weight of at least one compound of the formula II 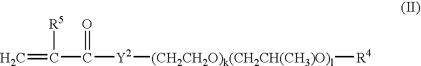
   in which
   the order of the alkylene oxide units is arbitrary,
   k and l, independently of one another, are an integer from 0 to 1000, where the sum of k and l is at least 5,
   R⁴ is hydrogen, C₁-C₃₀-alkyl, preferably C₁₂-C₂₆-alkyl
   R⁵ is hydrogen or C₁-C₈-alkyl,
   Y² is O or NR⁶, where R⁶ is hydrogen, C₁-C₃₀-alkyl or C₅-C₈-cycloalkyl,
iv) 0 to 20% by weight of at least one monomer which is different from, and copolymerizable with, components i) to iii), and
v) 0.1 to 3% by weight of at least one crosslinking monomer with at least two ethylenically unsaturated, nonconjugated double bonds.

In a further preferred embodiment, compositions in the form of a gel comprise at least one polyelectrolyte complex A) in which the carboxylic-acid-group-containing polymer A2) comprises, in copolymerized form,
i) 90 to 99.9% by weight of acrylic acid and/or methacrylic acid,
ii) 0 to 9.9% by weight of at least one monomer which is different from, and copolymerizable with, component i),
iii) 0.1 to 3% by weight of at least one crosslinking monomer with at least two ethylenically unsaturated, nonconjugated double bonds.

The above-described polyelectrolyte complexes A) are highly suitable for the preparation of cosmetic and pharmaceutical compositions. They are used here, for example, as polymeric film formers in preparations for body care, which involves the application of cosmetic preparations to keratin surfaces, such as skin, hair, nails, and also mouth care preparations. They can be used and formulated universally in a very wide variety of cosmetic preparations and are compatible with the customary components. For the same solids content, they generally exhibit considerably lower viscosities than aqueous preparations based on polymers known from the prior art. They thus permit the formulation of liquid to gel-like products with relatively high solids contents and are notable for improved conditioner properties.

Cosmetically Acceptable Carrier B)

The compositions according to the invention have a cosmetically or pharmaceutically acceptable carrier B) which is chosen from
i) water,
ii) water-miscible organic solvents, preferably C₂-C₄-alkanols, in particular ethanol
iii) oils, fats, waxes,
iv) esters of C₆-C₃₀-monocarboxylic acids with mono-, di or trihydric alcohols which are different from iii),
v) saturated acyclic and cyclic hydrocarbons,
vi) fatty acids
vii) fatty alcohols
viii) propellant gases
   and mixtures thereof.

The compositions according to the invention have, for example, an oil or fat component B) which is chosen from: hydrocarbons of low polarity, such as mineral oils; linear saturated hydrocarbons, preferably with more than 8 carbon atoms, such as tetradecane, hexadecane, octadecane etc.; cyclic hydrocarbons, such as decahydronaphthalene; branched hydrocarbons; animal and vegetable oils; waxes; wax esters; petroleum jelly; esters, preferably esters of fatty acids, such as, for example, the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkyl benzoates, benzyl benzoate; other cosmetic esters, such as fatty acid triglycerides, propylene glycol monolaurate, polyethylene glycol monolaurate, C₁₀-C₁₅-alkyl lactates, etc. and mixtures thereof.

Suitable silicone oils B) are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1 000 to 150 000 g/mol. Preferred cyclic siloxanes have 4- to 8-membered rings. Suitable cyclic siloxanes are commercially available, for example, under the name cyclomethicone.

Preferred oil or fat components B) are chosen from paraffin and paraffin oils; petroleum jelly; natural fats and oils, such as castor oil, soybean oil, groundnut oil, olive oil, sunflower oil, sesame oil, avocado oil, cocoa butter, almond oil, peach kernel oil, castor oil, cod-liver oil, lard, spermaceti, spermaceti oil, sperm oil, wheatgerm oil, macadamia nut oil, evening primrose oil, jojoba oil; fatty alcohols, such as lauryl alcohol, myristyl alcohol, cetyl alcohol, stearyl alcohol, oleyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candililla wax, spermaceti and mixtures of the abovementioned oil and fat components.

Suitable cosmetically and pharmaceutically compatible oil or fat components B) are described in Karl-Heinz Schrader, Grundlagen und Rezepturen der Kosmetika [Fundamentals and formulations of cosmetics], 2nd edition, Verlag Hüthig, Heidelberg, pp. 319-355, which is hereby incorporated by reference.

Suitable hydrophilic carriers B) are chosen from water, 1-, 2- or polyhydric alcohols with preferably 1 to 8 carbon atoms, such as ethanol, n-propanol, isopropanol, propylene glycol, glycerol, sorbitol, etc.

The cosmetic compositions according to the invention may be skin cosmetic, hair cosmetic, dermatological, hygienic or pharmaceutical compositions. On the basis of their film-forming properties, the above-described polyelectrolyte complexes A) are suitable in particular as additives for hair and skin cosmetics.

The compositions according to the invention are preferably in the form of a gel, foam, spray, ointment, cream, emulsion, suspension, lotion, milk or paste. If desired, liposomes or microspheres can also be used.

The cosmetically or pharmaceutically active compositions according to the invention can additionally comprise cosmetically and/or dermatologically active ingredients and auxiliaries.

The cosmetic compositions according to the invention preferably comprise at least one polyelectrolyte complex A), at least one carrier B) as defined above and at least one constituent different therefrom which is chosen from cosmetically active ingredients, emulsifiers, surfactants, preservatives, perfume oils, thickeners, hair polymers, hair and skin conditioners, graft polymers, water-soluble or dispersible silicone-containing polymers, light protection agents, bleaches, gel formers, care agents, colorants, tinting agents, tanning agents, dyes, pigments, consistency-imparting agents, humectants, refatting agents, collagen, protein hydrolyzates, lipids, antioxidants, antifoams, antistats, emollients and softeners.

Customary thickeners in such formulations are crosslinked polyacrylic acids and derivatives thereof, polysaccharides and derivatives thereof, such as xanthan gum, agar agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone. Preference is given to using nonionic thickeners.

Suitable cosmetically and/or dermatologially active ingredients are, for example, coloring active ingredients, skin and hair pigmentation agents, tinting agents, tanning agents, bleaches, keratin-hardening substances, antimicrobial active ingredients, light filter active ingredients, repellent active ingredients, substances with a hyperemic effect, substances with a keratolytic and keratoplastic effect, antidandruff active ingredients, antiphlogistics, substances with a keratinizing effect, active ingredients with an antioxidative effect or a free-radical scavenging effect, substances which wet the skin or retain moisture, refatting active ingredients, antierythematous or antiallergic active ingredients and mixtures thereof.

Active ingredients which tan the skin artifically and which are suitable for tanning the skin without natural or artificial irradiation with UV rays are, for example, dihydroxyacetone, alloxan and walnut shell extract. Suitable keratin-hardening substances are usually active ingredients as are also used in antiperspirants, such as, for example, potassium aluminum sulfate, aluminum hydroxychloride, aluminum lactate, etc. Antimicrobial active ingredients are used in order to destroy microorganisms or to inhibit their growth and thus serve both as preservatives and also as a deodorizing substance which reduces the formation or the intensity of body odor. These include, for example, customary preservatives known to the person skilled in the art, such as p-hydroxybenzoic esters, imidazolidinylurea, formaldehyde, sorbic acid, benzoic acid, salicylic acid, etc. Such deodorizing substances are, for example, zinc ricinoleate, triclosan, undecylenic acid alkylolamides, triethyl citrate, chlorhexidine etc. Suitable light filter active ingredients are substances which absorb UV rays in the UV-B- and/or UV-A region. Suitable UV filters are, for example, 2,4,6-triaryl-1,3,5-triazines in which the aryl groups may in each case carry at least one substituent which is preferably chosen from hydroxy, alkoxy, specifically methoxy, alkoxycarbonyl, specifically methoxycarbonyl and ethoxycarbonyl and mixtures thereof. Also suitable are p-aminobenzoates, cinnamates, benzophenones, camphor derivatives, and pigments which stop UV rays, such as titanium dioxide, talc and zinc oxide. Suitable repellent active ingredients are compounds which are able to drive away or keep certain animals, in particular insects, away from humans. These include, for example, 2-ethyl-1,3-hexanediol, N,N-diethyl-m-toluamide etc. Suitable substances with hyperemic activity, which stimulate blood flow through the skin are, for example, essential oils, such as dwarf pine, lavender, rosemary, juniperberry, roast chestnut extract, birch leaf extract, hayseed extract, ethyl acetate, camphor, menthol, peppermint oil, rosemary extract, eucalyptus oil, etc. Suitable keratolytic and keratoplastic substances are, for example, salicylic acid, calcium thioglycolate, thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiphlogistics, which counter skin irritations, are, for example, allantoin, bisabolol, Dragosantol, camomile extract, panthenol, etc.

The cosmetic compositions according to the invention can comprise, as cosmetic and/or pharmaceutical actice ingredient (and also in some instances as auxiliary), at least one cosmetically or pharmaceutically acceptable polymer which differs from the polymers which form the polyelectrolyte complex used according to the invention. These include, quite generally, cationic, amphoteric and neutral polymers.

Suitable polymers are, for example, cationic polymers with the INCI name Polyquaternium, e.g. copolymers of vinylpyrrolidone/N-vinylimidazolium salts (Luviquat®) FC, Luviquat® HM, Luviquat®MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimida-zolium salts (Luviquat® Hold); cationic cellulose derivatives (Polyquaternium-4 and -10), acrylamido copolymers (Polyquaternium-7) and chitosan. Suitable cationic (quaternized) polymers are also Merquat® (polymer based on dimethyldiallylammonium chloride), Gafquat® (quaternary polymers which are formed by the reaction of polyvinylpyrrolidone with quaternary ammonium compounds), Polymer JR (hydroxyethylcellulose with cationic groups) and plant-based cationic polymers, e.g. guar polymers, such as the Jaguar® grades from Rhodia.

Further suitable polymers are also neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyethylenimines and salts thereof, polyvinylamines and salts thereof, cellulose derivatives, polyaspartic acid salts and derivatives. These include, for example, Luviflex® Swing (partially hydrolyzed copolymer of polyvinyl acetate and polyethylene glycol, BASF).

Suitable polymers are also nonionic, water-soluble or water-dispersible polymers or oligomers, such as polyvinylcaprolactam, e.g. Luviskol® Plus (BASF) or polyvinylpyrrolidone and copolymers thereof, in particular with vinyl esters, such as vinyl acetate, e.g. Luviskol® VA 37 (BASF); polyamides, e.g. based on itaconic acid and aliphatic diamines, as are described, for example, in DE-A43 33 238.

Suitable polymers are also amphoteric or zwitterionic polymers, such as the octyl acrylamide/methyl methacrylate/tert-butylaminoethyl methacrylate/2-hydroxypropyl methacrylate copolymers which are available commercially under the name Amphomer® (National Starch) and zwitterionic polymers as are disclosed, for example, in German patent applications DE 39 29 973, DE 21 50 557, DE 28 17 369 and DE 37 08 451. Acrylamidopropyltrimethylammonium chloride/acrylic acid or methacrylic acid copolymers and alkali metal and ammonium salts thereof are preferred zwitterionic polymers. Further suitable zwitterionic polymers are methacroylethylbetaine/methacrylate copolymers which are available commercially under the name Amersette® (AMERCHOL), and copolymers of hydroxyethyl methacrylate, methyl methacrylate, N,N-dimethylaminoethyl methacrylate and acrylic acid (Jordapon®).

Suitable polymers are also nonionic, siloxane-containing, water-soluble or -dispersible polymers, e.g. polyethersiloxanes, such as Tegopren® (Goldschmidt) or Belsil® (Wacker).

The formulation base of pharmaceutical compositions according to the invention preferably comprises pharmaceutically acceptable auxiliaries. Pharmaceutically acceptable auxiliaries are the auxiliaries which are known for use in the field of pharmacy, food technology and related fields, in particular those listed in the relevant pharmacopoeia (e.g. DAB Ph. Eur. BP NF), and other auxiliaries whose properties do not preclude a physiological application.

Suitable auxiliaries may be: lubricants, wetting agents, emulsifying and suspending agents, preservatives, antioxidants, antiirritative substances, chelating agents, emulsion stabilizers, film formers, gel formers, odor-masking agents, resins, hydrocolloids, solvents, solubility promoters, neutralizing agents, permeation accelerators, pigments, quaternary ammonium compounds, refatting and superfatting agents, ointment bases, cream bases or oil bases, silicone derivatives, stabilizers, sterilizing agents, propellants, drying agents, opacifiers, thickeners, waxes, softeners, white oils. Formulation in this regard is based on specialist knowledge, as given, for example, in Fiedler, H. P. Lexikon der Hilfsstoffe für Pharmazie, Kosmetik und angrenzende Gebiete [Lexicon of auxiliaries for pharmacy, cosmetics and related fields], 4th ed., Aulendorf: ECV-Editio-Kantor-Verlag, 1996.

To prepare the dermatological compositions according to the invention, the active ingredients can be mixed or diluted with a suitable auxiliary (excipient). Excipients may be solid, semisolid or liquid materials which can also serve as vehicles, carriers or medium for the active ingredient. The admixing of further auxiliaries is carried out, where desired, in the manner known to the person skilled in the art. In addition, the polymers P) and dispersions Pd) are suitable as auxiliaries in pharmacy, preferably as a or in coating(s) or binder(s) for solid drug forms. They can also be used in creams and as tablet coatings and tablet binders.

According to a preferred embodiment, the compositions according to the invention are a skin-cleansing composition.

Preferred skin-cleansing compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, shower and bath preparations, such as washing lotions, shower baths and shower gels, foam baths, oil baths and scrub preparations, shaving foams, lotions and creams.

According to a further preferred embodiment, the compositions according to the invention are cosmetic compositions for the care and protection of the skin, nailcare compositions or preparations for decorative cosmetics.

Suitable skin cosmetic compositions are, for example, face tonics, face masks, deodorants and other cosmetic lotions. Compositions for use in decorative cosmetics include, for example, concealer pencils, stage makeup, mascara and eyeshadows, lipsticks, kohl pencils, eyeliners, blushers, powders and eyebrow pencils.

Furthermore, the polyelectrolyte complexes A) can be used in nose strips for pore cleansing, in antiacne compositions, repellents, shaving compositions, hair-removal compositions, personal hygiene compositions, footcare compositions, and in babycare.

The skincare compositions according to the invention are, in particular, W/O or O/W skin creams, day and night creams, eye creams, face creams, antiwrinkle creams, moisturizing creams, bleaching creams, vitamin creams, skin lotions, care lotions and moisturizing lotions.

Skin cosmetic and dermatological compositions based on the above-described polyelectrolyte complexes A) exhibit advantageous effects. The polymers can, inter alia, contribute to the moisturization and conditioning of the skin and to an improvement in the feel of the skin. The polymers can also act as thickeners in the formulations. By adding the polymers according to the invention, it is possible to achieve a considerable improvement in skin compatibility in certain formulations.

Skin cosmetic and dermatological compositions preferably comprise at least one polyelectrolyte complex A) in an amount of from about 0.001 to 30% by weight, preferably 0.01 to 20% by weight, very particularly preferably 0.1 to 12% by weight, based on the total weight of the composition.

Light protection agents based on the polyelectrolyte complexes A) in particular have the property of increasing the residence time of the UV-absorbing ingredients compared with customary auxiliaries such as polyvinylpyrrolidone.

Depending on the field of use, the compositions according to the invention can be applied in a form suitable for skincare, such as, for example, in the form of a cream, foam, gel, pencil, mousse, milk, spray (pump spray or spray containing propellant) or lotion.

As well as comprising the polyelectrolyte complexes A) and suitable carriers, the skin cosmetic preparations can also comprise further active ingredients and auxiliaries customary in skin cosmetics and as described above. These include, preferably, emulsifiers, preservatives, perfume oils, cosmetic active ingredients, such as phytantriol, vitamin A, E and C, retinol, bisabolol, panthenol, light protection agents, bleaches, colorants, tinting agents, tanning agents, collagen, protein hydrolyzates, stabilizers, pH regulators, dyes, salts, thickeners, gel formers, consistency-imparting agents, silicones, humectants, refatting agents and further customary additives.

Preferred oil and fat components of the skin cosmetic and dermatological compositions are the abovementioned mineral and synthetic oils, such as, for example, paraffins, silicone oils and aliphatic hydrocarbons with more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, petroleum jelly, hydrogenated lanolin and acetylated lanolin, and mixtures thereof.

The polymers according to the invention can also be mixed with conventional polymers where specific properties are to be set.

To set certain properties, such as, for example, improving the feel to the touch, the spreading behavior, the water resistance and/or the binding of active ingredients and auxiliaries, such as pigments, the skin cosmetic and dermatological preparations can additionally also comprise conditioning substances based on silicone compounds. Suitable silicone compounds are, for example, polyalkylsiloxanes, polyarylsiloxanes, polyarylalkylsiloxanes, polyether siloxanes or silicone resins.

The cosmetic or dermatological preparations are prepared by customary processes known to the person skilled in the art.

The cosmetic and dermatological compositions are preferably in the form of emulsions, in particular in the form of water-in-oil (W/O) or oil-in-water (O/W) emulsions. It is, however, also possible to choose other types of formulation, for example hydrodispersions, gels, oils, oleogels, multiple emulsions, for example in the form of W/O/W or O/W/O emulsions, anhydrous ointments or ointment bases, etc.

The emulsions are prepared by known methods. Apart from at least one polyelectrolyte complex A), the emulsions usually comprise customary constituents, such as fatty alcohols, fatty acid esters and in particular fatty acid triglycerides, fatty acids, lanolin and derivatives thereof, natural or synthetic oils or waxes and emulsifiers in the presence of water. The choice of emulsion type-specific additives and the preparation of suitable emulsions is described, for example, in Schrader, Grundlagen und Rezepturen der Kosmetika [Fundamentals and formulations of cosmetics], Hüthig Buch Verlag, Heidelberg, 2nd edition, 1989, third part, which is hereby expressly incorporated by reference.

A suitable emulsion, e.g. for a skin cream etc., generally comprises an aqueous phase which is emulsified by means of a suitable emulsifier system in an oil or fatty phase. A polyelectrolyte complex A) can be used to prepare the aqueous phase.

Preferred fatty components which may be present in the fatty phase of the emulsions are: hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karité oil, hoplostethus oil; mineral oils whose distillation start-point under atmospheric pressure is about 250° C. and whose distillation end-point is 410° C., such as, for example, vaseline oil; esters of saturated or unsaturated fatty acids, such as alkyl myristates, e.g. i-propyl, butyl or cetyl myristate, hexadecyl stearate, ethyl or isopropyl palmitate, octanoic or decanoic acid triglycerides and cetyl ricinoleate.

The fatty phase can also comprise silicone oils soluble in other oils, such as dimethylpolysiloxane, methylphenylpolysiloxane and the silicone glycol copolymer, fatty acids and fatty alcohols.

As well as the polyelectrolyte complexes A), it is also possible to use waxes, such as, for example, carnauba wax, candililla wax, beeswax, microcrystalline wax, ozocerite wax and Ca, Mg and Al oleate, myristates, linoleates and stearates.

In addition, an emulsion according to the invention may be present in the form of a O/W emulsion. An emulsion of this type usually comprises an oil phase, emulsifiers which stabilize the oil phase in the water phase, and an aqueous phase which is usually present in thickened form. Suitable emulsifiers are preferably O/W emulsifiers, such as polyglycerol esters, sorbitan esters and partially esterified glycerides.

According to a further preferred embodiment, the compositions according to the invention are a shower gel, a shampoo formulation or a bath preparation.

Such formulations comprise at least one polyelectrolyte complex A) and also usually anionic surfactants as base surfactants and amphoteric and/or nonionic surfactants as cosurfactants. Further suitable active ingredients and/or auxiliaries are generally chosen from lipids, perfume oils, dyes, organic acids, preservatives and antioxidants, and also thickeners/gel formers, skin conditioning agents and humectants.

These formulations preferably comprise 2 to 50% by weight, preferably 5 to 40% by weight, particularly preferably 8 to 30% by weight, of surfactants, based on the total weight of the formulation.

All anionic, neutral and amphoteric or cationic surfactants customarily used in body-cleansing compositions can be used in the washing, showering and bathing preparations.

Suitable anionic surfactants are, for example, alkyl sulfates, alkyl ether sulfates, alkylsulfonates, alkylarylsulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

These include, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or alkyl amphopropionates, alkyl amphodiacetates or alkyl amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. Also suitable are alkylamine oxides, mono- or dialkylalkanolamides, fatty acid esters of polyethylene glycols, ethoxylated fatty acid amides, alkyl polyglycosides or sorbitan ether esters.

The washing, shower and bath preparations can also comprise customary cationic surfactants, such as, for example, quaternary ammonium compounds, for example cetyltrimethylammonium chloride.

In addition, the shower gel/shampoo formulations may comprise thickeners, such as, for example, sodium chloride, PEG-55, propylene glycol oleate, PEG-120-methylglucose dioleate and others, and also preservatives, further active ingredients and auxiliaries and water.

According to a further preferred embodiment, the compositions according to the invention are a hair-treatment composition.

Hair-treatment compositions according to the invention preferably comprise at least one polyelectrolyte A) in an amount in the range from about 0.1 to 30% by weight, preferably 0.5 to 20% by weight, based on the total weight of the composition.

The hair-treatment compositions according to the invention are preferably in the form of a setting foam, hair mousse, hair gel, shampoo, hairspray, hair foam, end fluid, neutralizing agent for permanent waves, hair colorant and bleach or hot-oil treatment. Depending on the field of use, the hair cosmetic preparations can be applied in the form of an (aerosol) spray, (aerosol) foam, gel, gel spray, cream, lotion or wax. Hairsprays include both aerosol sprays and also pump sprays without propellant gas. Hair foams include both aerosol foams and also pump foams without propellant gas. Hairsprays and hair foams preferably comprise predominantly or exclusively water-soluble or water-dispersible components. If the compounds used in the hairsprays and hair foams according to the invention are water-dispersible, they can be applied in the form of aqueous microdispersions with particle diameters of usually 1 to 350 nm, preferably 1 to 250 nm. The solids contents of these preparations here are usually in a range from about 0.5 to 20% by weight. These microdispersions generally require no emulsifiers or surfactants for their stabilization.

In a preferred embodiment, the hair cosmetic formulations according to the invention comprise

a) 0.05 to 20% by weight of at least one polyelectrolyte complex A),

b) 20 to 99.95% by weight of water and/or alchol,

c) 0 to 50% by weight of at least one propellant gas,

d) 0 to 5% by weight of at least one emulsifier,

e) 0 to 3% by weight of at least one thickener, and

f) up to 25% by weight of further constituents.

Alcohol is understood as meaning all alcohols customary in cosmetics, e.g. ethanol, isopropanol, n-propanol.

Further constituents are understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances, such as panthenol, collagen, vitamins, protein hydrolyzates, alpha- and beta-hydroxycarboxylic acids, stabilizers, pH regulators, dyes, viscosity regulators, gel formers, salts, humectants, refatting agents, complexing agents and further customary additives.

These also include all styling and conditioning polymers known in cosmetics which may be used in combination with the polymers according to the invention if very particular properties are to be set.

Suitable conventional hair cosmetic polymers are, for example, the abovementioned cationic, anionic, neutral, nonionic and amphoteric polymers, which are hereby incorporated by reference.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyether siloxanes, silicone resins or dimethicone copolyols (CTFA) and aminofunctional silicone compounds such as amodimethicone (CTFA).

The polymers according to the invention are suitable in particular as setting agents in hairstyling preparations, in particular hairsprays (aerosol sprays and pump sprays without propellant gas) and hair foams (aerosol foams and pump foams without propellant gas).

In a preferred embodiment, spray preparations comprise

a) 0.1 to 10% by weight of at least one polyelectrolye complex A),

b) 20 to 99.9% by weight of water and/or alcohol,

c) 0 to 70% by weight of at least one propellant,

d) 0 to 20% by weight of further constituents.

Propellants are the propellants customarily used for hairsprays or aerosol foams.

Preference is given to mixtures of propane/butane, pentane, dimethyl ether, 1,1-difluoroethane (HFC-152 a), carbon dioxide, nitrogen or compressed air.

A formulation for aerosol hair foams preferred according to the invention comprises

a) 0.1 to 10% by weight of at least one polyelectrolyte complex A),

b) 55 to 99.8% by weight of water and/or alcohol,

c) 5 to 20% by weight of a propellant,

d) 0.1 to 5% by weight of an emulsifier,

e) 0 to 10% by weight of further constituents.

Emulsifiers which may be used are all emulsifiers customarily used in hair foams. Suitable emulsifiers may be nonionic, cationic or anionic or amphoteric.

Examples of nonionic emulsifiers (INCI nomenclature) are Laureths, e.g. Laureth-4; Ceteths, e.g. Ceteth-1, polyethylene glycol cetyl ether; Ceteareths, e.g. Ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methylsulfate, Quaternium-1 to x (INCI).

Anionic emulsifiers can, for example, be chosen from the group of alkylsulfates, alkyl ether sulfates, alkylsulfonates, alkylarylsulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition:

a) 0.1 to 10% by weight of at least one polyelectrolyte complex A),

b) 80 to 99.85% by weight of water and/or alcohol,

c) 0 to 3% by weight, preferably 0.05 to 10% by weight of a gel former,

d) 0 to 20% by weight of further constituents.

The polyelectrolyte complexes A) used according to the invention generally already have a “self-thickening” action, meaning that in many cases the use of gel formers can be dispensed with in the preparation of gels. However, their use may be advantageous for establishing specific rheological or other performance properties of the gels. Gel formers which can be used are all gel formers customary in cosmetics. These include slightly crosslinked polyacrylic acid, for example Carbomer (INCI), cellulose derivatives, e.g. hydroxypropylcellulose, hydroxyethylcellulose, cationically modified celluloses, polysaccharides, e.g. xanthan gum, caprylic/capric triglyceride, sodium acrylate copolymers, Polyquaternium-32 (and) Paraffinum Liquidum (INCI), sodium acrylate copolymers (and) Paraffinum Liquidum (and) PPG-1 Trideceth-6, Acrylamidopropyl Trimonium Chloride/Acrylamide Copolymers, Steareth-10 Allyl Ether Acrylates Copolymer, Polyquaternium-37 (and) Paraffinum Liquidum (and) PPG-1 Trideceth-6, Polyquaternium 37 (and) Propylene Glycol Dicaprate Dicaprylate (and) PPG-1 Trideceth-6, Polyquaternium-7, Polyquaternium-44.

The polyelectrolyte complexes A) according to the invention can be used in cosmetic preparations as conditioning agents.

The polyelectrolyte complexes A) according to the invention can preferably be used in shampoo formulations as setting and/or conditioning agents. Preferred shampoo formulations comprise

a) 0.05 to 10% by weight of at least one polyelectrolyte complex A),

b) 25 to 94.95% by weight of water,

c) 5 to 50% by weight of surfactants,

c) 0 to 5% by weight of a further conditioning agent,

d) 0 to 10% by weight of further cosmetic constituents.

All anionic, neutral, amphoteric or cationic surfactants customarily used in shampoos can be used in the shampoo formulations.

Suitable anionic surfactants are, for example, alkyl sulfates, alkyl ether sulfates, alkylsulfonates, alkylarylsulfonates, alkyl succinates, alkyl sulfosuccinates, N-alkoylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

For example, sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecylbenzenesulfonate are suitable.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. Also suitable are alkylamine oxides, mono- or dialkylalkanolamides, fatty acid esters of polyethylene glycols, alkylpolyglycosides or sorbitan ether esters.

Furthermore, the shampoo formulations can comprise customary cationic surfactants, such as, for example, quaternary ammonium compounds, for example cetyltrimethylammonium chloride.

In the shampoo formulations, customary conditioning agents can be used in combination with the polyelectrolyte complexes A) to achieve certain effects. These include, for example, the abovementioned cationic polymers with the INCI name Polyquaternium, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamide copolymers (Polyquaternium-7). It is also possible to use protein hydrolyzates, and conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and aminofunctional silicone compounds, such as amodimethicone (CTFA). In addition, cationic guar derivatives, such as guar hydroxypropyltrimonium chloride (INCI) can be used.

The invention further provides the use of a polyelectrolyte complex, as defined above, as auxiliary in pharmacy, preferably as or in (a) coating(s) for solid drug forms, for modifying rheological properties, as surface-active complex, as or in (an) adhesive(s), and as or in (a) coating(s) for the textile, paper, printing and leather industry.

The invention is illustrated in more detail by reference to the following non limiting examples.

### EXAMPLES

1. Preparation of Copolymers (Solution Polymerization)

### Example 23

### Copolymer of VP/VI/MAM

| | |
|---|---|
| Feed 1: | Monomer mixture of: |
| 120 g | Vinylpyrrolidone |
| 40 g | Vinylimidazol |
| 467 g | Methacrylamide (15% strength solution) |
| Feed 2: | Initiator solution of: |
| 1.2 g | Wako V 50 [2,2′-azobis(2-amidinopropane) dihydrochloride] and |
| 30 g | Water |
| Feed 3: | Initiator solution of: |
| 0.6 g | Wako V 50 [2,2′-azobis(2-amidinopropane) dihydrochloride] and |
| 15 g | Water |

30 g of Feed 1, 2 g of Feed 2 and 160 g of water were initially introduced into a stirred apparatus with reflux condenser, internal thermometer and 3 separate feed devices and the mixture was heated to about 65° C. with stirring. Following the start of polymerization, recognizable from the viscosity starting to increase, at 65° C., the remainder of Feed 1 was added over the course of 4 h and the remainder of Feed 2 over 5 h, the internal temperature being increased to about 68° C. When the addition was complete, the reaction mixture was further stirred for about 2 h at 70° C. Feed 3 was then added over the course of 30 minutes at a temperature of 70° C. and the polymer solution was then further after-polymerized for about 2 h at a temperature of about 80° C. The polymer solution was treated for 2 h with steam. This gave an about 30% strength polymer solution.

For the stabilization, the solution was treated with 100 ppm of Euxyl k100 from Schulke & Mayr (5-chloro-2-methyl-3-(2)-isothiazolon/2-methyl-3-(2H)-isothiazolon/benzyl alcohol).

Pulverulent products were obtained by spray-drying or freeze-drying.

The polymers 1 to 35 in table 1 were prepared analogously.

TABLE 1

| Ex. | | | | | | DMAP | | |
|---|---|---|---|---|---|---|---|---|
| No. | VP | VCap | MAM | DMAA | HEMA | MAM | QVI | VI |
| C1* | 100 | — | — | — | — | — | — | — |
| C2* | 90 | — | — | — | — | 10 | — | — |
| C3* | 90 | — | — | — | — | — | 10 | — |
| 1 | 90 | — | — | — | — | — | — | 10 |
| 2 | 80 | — | — | — | — | — | — | 20 |
| 3 | 75 | — | — | — | — | — | — | 25 |
| 4 | 70 | — | — | — | — | — | — | 30 |
| 5 | 50 | — | — | — | — | — | — | 50 |
| 6 | 60 | 20 | — | — | — | — | — | 20 |
| 7 | 60 | — | 20 | — | — | — | — | 20 |
| 8 | 60 | — | — | 20 | — | — | — | 20 |
| 9 | 60 | — | — | — | 20 | — | — | 20 |
| 10 | 60 | 25 | — | — | — | — | — | 15 |
| 11 | 60 | — | 25 | — | — | — | — | 15 |
| 12 | 60 | — | — | 25 | — | — | — | 15 |
| 13 | 60 | — | — | — | 25 | — | — | 15 |
| 14 | 60 | 30 | — | — | — | — | — | 10 |
| 15 | 60 | — | 30 | — | — | — | — | 10 |
| 16 | 60 | — | — | 30 | — | — | — | 10 |
| 17 | 55 | 35 | — | — | — | — | — | 10 |
| 18 | 55 | — | 35 | — | — | — | — | 10 |
| 19 | 55 | — | — | 35 | — | — | — | 10 |
| 20 | 55 | 38 | — | — | — | — | — | 7 |
| 21 | 55 | — | 38 | — | — | — | — | 7 |
| 22 | 60 | 35 | — | — | — | — | — | 5 |
| 23 | 60 | — | 35 | — | — | — | — | 5 |
| 24 | 60 | — | — | 35 | — | — | — | 5 |
| 25 | 60 | 20 | — | — | 15 | — | — | 5 |
| 26 | 60 | — | 20 | — | 15 | — | — | 5 |
| 27 | 60 | — | — | 20 | 15 | — | — | 5 |
| 28 | 57 | 40 | — | — | — | — | — | 3 |
| 29 | 57 | — | 40 | — | — | — | | 3 |
| 30 | 57 | — | — | 20 | 20 | — | | 3 |
| 31 | 55 | 35 | — | — | — | — | 5 | 5 |
| 32 | 55 | — | 35 | — | — | — | 5 | 5 |
| 33 | 55 | — | 30 | — | — | — | 5 | 10 |
| 34 | 55 | 25 | — | — | — | — | 10 | 10 |
| 35 | 55 | — | 25 | — | — | — | 10 | 10 |

*= Comparative examples
VP = Vinylpyrrolidone
VCap = Vinylcaprolactam
MAM = Methacrylamide
DMAA = N,N-dimethylacrylamide
HEMA = Hydroxyethyl methacrylate
DMAPMAM = Dimethylaminopropylmethacrylamide
VI = Vinylimidazol
QVI = N-vinylimidazolium methylsulfate

The copolymers containing cationogenic groups in table 1 were reacted firstly with hydrophobic modified crosslinked polyacrylates (Carbopol® Ultrez 21 from Noveon) and secondly with acrylic acid/Beheneth-25 methacrylate copolymers (Aculyn® 28 from Rohm und Haas) to give polyelectrolyte complexes and formulated to give gels. The performance properties are shown in table 2 below.

The grade awarded was determined according to the following scale: 1 very good, 2 satisfactory, 3 unsatisfactory

TABLE 2

| | Gel with Aculyn 28 (0.5%) | | | | Gel with Ultrez 21 (0.5%) | | | |
|---|---|---|---|---|---|---|---|---|
| Ex. | Styling/clarity/ | | | | Styling/clarity/ | | | |
| No. | tackiness/viscosity | | | | tackiness/viscosity | | | |
| C1 | 2 | 1 | 2 | moderate | 2 | 1 | 2 | solid |
| C2 | 2 | 3 | 2 | inhomog. | 2 | 3 | 2 | inhomog. |
| C3 | 2 | 1-2 | 2-3 | moderate | 2 | 1-2 | 2-3 | moderate |
| 1 | 1-2 | 1 | 2 | solid | 1 | 1-2 | 2 | solid |
| 2 | 1 | 1-2 | 2 | solid | | | | |
| 3 | 1 | 1-2 | 1-2 | solid | | | | |
| 4 | 1 | 1-2 | 1-2 | solid | | | | |
| 5 | 1 | 2 | 1 | solid | | | | |
| 6 | 1 | 1-2 | 1-2 | solid | | | | |
| 7 | 1 | 1-2 | 1 | solid | | | | |
| 8 | 1 | 1-2 | 1-2 | solid | | | | |
| 9 | 1 | 2 | 1 | solid | | | | |
| 10 | 1 | 1 | 1-2 | solid | | | | |
| 11 | 1 | 1 | 1 | solid | | | | |
| 12 | 1 | 1-2 | 1-2 | solid | | | | |
| 13 | 1 | 2 | 1 | solid | | | | |
| 14 | 1 | 1 | 1-2 | solid | | | | |
| 15 | 1 | 1 | 1 | solid | 1 | 1-2 | 1 | solid |
| 16 | 1 | 1 | 1-2 | solid | | | | |
| 17 | 1 | 1 | 1-2 | solid | 1-2 | 2 | 1-2 | solid |
| 18 | 1 | 1 | 1 | solid | 1 | 1-2 | 1 | solid |
| 19 | 1 | 1 | 2 | solid | | | | |
| 20 | 1 | 1 | 1-2 | solid | | | | |
| 21 | 1 | 1 | 1 | solid | | | | |
| 22 | 1 | 1 | 1-2 | solid | | | | |
| 23 | 1 | 1 | 1 | solid | 1 | 1 | 1 | solid |
| 24 | 1 | 1 | 2 | solid | | | | |
| 25 | 1 | 1-2 | 1-2 | Solid | 1 | 2 | 1-2 | solid |
| 26 | 1 | 1 | 1-2 | solid | | | | |
| 27 | 1 | 1 | 1-2 | solid | | | | |
| 28 | 1 | 1 | 1-2 | solid | 1-2 | 1 | 1-2 | solid |
| 29 | 1 | 1 | 1 | solid | 1 | 1 | 1 | solid |
| 30 | 2 | 1-2 | 1 | solid | | | | |
| 31 | 1 | 1-2 | 1-2 | solid | 1 | 2 | 1-2 | solid |
| 32 | 1 | 1 | 1 | solid | 1 | 1-2 | 1 | solid |
| 33 | 1 | 1 | 1-2 | solid | | | | |
| 34 | 2 | 2 | 2 | solid | | | | |
| 35 | 1-2 | 2 | 1-2 | solid | | | | |

2. Application Examples
A) Use in hair cosmetics:

1) Hair Gels with an Anionic Thickener: Examples No. 1-35

| | [%] |
|---|---|
| Phase 1: | |
| Polymer 1-35 (30% strength aqueous solution) | 10.0 |
| Water, dist. | 39.0 |
| Aminomethylpropanol (38% strength solution) | 1.0 |
| Further additive: preservative, soluble | |
| ethoxylated silicone, perfume | |
| Phase 2: | |
| Aculyn 28 (1% strength aqueous suspension) | 50.0 |

Preparation: Phases 1 and 2 are weighed in separately and homogenized. Phase 2 is stirred slowly into Phase 1. A clear, solid gel is formed.

2) “Wet-Look Hair Gels”: Examples No. 36-70

| | [%] |
|---|---|
| Phase 1: | |
| Polymer 1-35 (30% strength aqueous solution) | 10.0 |
| Water, dist.. | 34.0 |
| Glycerol | 5.0 |
| Aminomethylpropanol (38% strength solution) | 1.0 |
| Further additive: preservative, soluble | |
| ethoxylated silicone, perfume | |
| Phase 2: | |
| Aculyn 28 (1% strength aqueous suspension) | 50.0 |

Preparation: Phases 1 and 2 are weighed in separately and homogenized. Phase 2 is slowly stirred into Phase 1. A clear, solid gel is formed.

3) Hair Gels with Ultrez 21: Examples No. 71-80

| | [%] | CTFA |
|---|---|---|
| Phase 1: | | |
| Polymer 1/15/17/18/23/25/28/29/31/32 | 10.0 | |
| (30% strength aqueous solution) | | |
| Water, dist. | 40.0 | |
| Triethanolamine | 0.5 | |
| Further additive: preservative, soluble | | |
| ethoxylated silicone, perfume | | |
| Phase 2: | | |
| Ultrez 21 (1% strength aquous suspension) | 49.5 | Carbomer |

Preparation: Phases 1 and 2 were weighed in separately and homogenized. Phase 2 is stirred slowly into Phase 1. A virtually clear, solid gel is formed.

4) Liquid Hair Gels: Examples No. 81-115

| | [%] | CTFA |
|---|---|---|
| Polymer 1-35 (30% strength aqueous solution) | 5.0 | |
| Glycerol | 3.0 | |
| Natrosol 250 L (1% strength aqueous solution) | 30.0 | Hydroxyethyl- |
| | | cellulose |
| | | (Hercules) |
| Luviflex Soft (10% strength aqueous solution, | 15.0 | Acrylate |
| pH = 7) | | copolymer |
| | | (BASF) |
| Water, dist. | 47.0 | |
| Further additive: preservative, soluble | | |
| ethoxylated silicone, perfume | | |

Preparation: Weigh in and homogenize slowly at room temperature with stirring.

5) Aqueous Hand Pumped Spray: Examples No. 116-150

| | [%] | CTFA |
|---|---|---|
| Polymer 1-35 (30% strength aqueous solution) | 5.0 | |
| Luviset ® PUR (30% strength water/ethanol | 5.0 | Polyurethane-1 |
| solution) | | (BASF) |
| Glycerol | 3.0 | |
| Water, dist. | 87.0 | |
| Further additive: preservative, soluble | | |
| ethoxylated silicone, perfume | | |

Preparation: Weigh in and homogenize slowly at room temperature with stirring.

6) Aerosol Hair Spray (VOC 55): Examples No. 151-160

| | [%] |
|---|---|
| Polymer 1/6/8/10/13/14/17/20/22/25 | 2.0 |
| (30% strength solution) | |
| Luviset ® PUR (30% strength water/ethanol solution) | 8.0 |
| Water | 35.5 |
| Dimethyl ether | 30.0 |
| Ethanol | 24.5 |
| Further additive: preservatives, soluble | |
| ethoxylated silicone, perfume, defoamer | |

7) Setting Foam: Examples No. 161-180

| | [%] |
|---|---|
| Polymer No. 1/2/6/7/10/11/14/15/17/18/ | 5.0 |
| 21-23/28/29/31-35 | |
| (30% strength aqueous solution) | |
| Cremophor A 25 (Ceteareth 25/BASF) | 0.2 |
| Comperlan KD (Coamide DEA/Henkel) | 0.1 |
| Water, dists. | 74.7 |
| Dimethyl ether | 10.0 |
| Further additive: perfume, preservative | |

Preparation: Weigh in and dissolve with stirring, bottle and add propellant gas.

8) Shampoo: Examples No. 181-190

| Conditioner Shampoo: | [%] |
|---|---|
| A) Texapon NSO 28% strength (Sodium laureth sulfate/Henkel) | 50.0 |
| Comperlan KD (Coamide DEA/Henkel) | 1.0 |
| Polymer 7/11/15/18/21/31-35 | 3.0 |
| (30% strength aqueous solution) | |
| Water | 17.0 |
| q.s. perfume oil | |
| B) Water | 27.5 |
| Sodium chloride | 1.5 |
| q.s. preservative | |

Preparation: Weigh in and dissolve Phases A and B separately with stirring and mix. Slowly stir Phase B into the Phase A.
B) Use in Skin Cosmetics:

9) Standard O/W Cream: Examples No. 191-210

| Oil phase: | % | CTFA Name |
|---|---|---|
| Cremophor A6 | 3.5 | Ceteareth-6 (and) Stearyl Alcohol |
| Cremophor A25 | 3.5 | Ceteareth-25 |
| Glycerol monostearate s.e. | 2.5 | Glyceryl Stearate |
| Paraffin oil | 7.5 | Paraffin Oil |
| Cetyl alcohol | 2.5 | Cetyl Alcohol |
| Luvitol EHO | 3.2 | Cetearyl Octanoate |
| Vitamin E acetate | 1.0 | Tocopheryl Acetate |
| Nip-Nip | 0.1 | Methyl and Propyl 4-hydroxy- |
| | | benzoate (7:3) |

| Water-Phase: | % | CTFA Name |
|---|---|---|
| Polymer No. 1/2/6/7/10/11/14/15/17/18/ | 3.0 | |
| 21-23/28/29/31-35 | | |
| (30% strength aqueous solution) | | |
| Water | 74.6 | Water |
| 1,2-Propylene glycol | 1.5 | Propylene glycol |
| Germall II | 0.1 | Imidazolidinylurea |

Preparation: Weigh in and homogenize the oil phase and water phase separately with stirring at a temperature of about 80° C. Slowly stir the water phase into the oil phase. Cool slowly with stirring to RT.

10) Day Lotion: Examples No. 211-230

| Oil phase: | % | CTFA Name |
|---|---|---|
| Cremophor A6 | 1.5 | Ceteareth-6 (and) Stearyl alcohol |
| Cremophor A25 | 1.5 | Ceteareth-25 |
| Glycerol monostearate s.e. | 5.0 | Glycerol stearate |
| Uvinul MS 40 | 0.5 | Benzophenon-4 |
| Paraffin oil | 3.5 | Paraffin oil |
| Cetyl alcohol | 0.5 | Cetyl alcohol |
| Luvitol EHO | 10.0 | Cetearyl octanoat |
| D-panthenol 50 P | 3.0 | Panthenol and Propylene glycol |
| Vitamin E acetate | 1.0 | Tocopheryl acetate |
| Tegiloxan 100 | 0.3 | Dimethicone |
| Nip-Nip | 0.1 | Methyl and Propyl 4- |
| | | hydroxybenzoate (7:3) |

| Water-Phase: | % | |
|---|---|---|
| Polymer No. 1/2/6/7/10/11/14/15/17/18/ | 1.5 | |
| 21-23/28/29/31-35 | | |
| (30% strength aqueous solution) | | |
| Water | 70.0 | Water |
| 1,2-Propylene glycol | 1.5 | Propylene glycol |
| Germall II | 0.1 | Imidazolidinylurea |

Preparation: Weigh in and homogenize the oil phases and water phase separately with stirring at a temperature of about 80° C. Slowing stir the water phase into the oil phase. Slowly cool to room temperature with stirring.

1. A cosmetic or pharmaceutical composition comprising
A) at least one water-soluble or water-dispersible polyelectrolyte complex comprising
A1) at least one water-soluble or water-dispersible copolymer with cationogenic groups which comprises, in copolymerized form, vinylimidazole and/or a derivative thereof and at least one further monomer copolymerizable therewith, and
A2) at least one acid-group-containing polymer, and
B) at least one cosmetically acceptable carrier.

2. A composition as claimed in claim 1, where the copolymer A1) comprises
a) vinylimidazole and/or a derivative thereof, and
b) at least one N-vinyllactam.

3. A composition as claimed in claim 2, where the copolymer A1) additionally comprises, in copolymerized form, at least one nonionic water-soluble monomer c) which is different from, and copolymerizable with, components a) and b).

4. A composition as claimed in claim 3, where monomer c) is chosen from N-vinylamides of saturated C₁-C₈-monocarboxylic acids, primary amides of α,β-ethylenically unsaturated monocarboxylic acids and the N-alkyl and N,N-dialkyl derivatives thereof which, in addition to the carbonyl carbon atom of the amide group, have at most 8 further carbon atoms, esters of α,β-ethylenically unsaturated mono- and dicarboxylic acids with diols, amides of α,β-ethylenically unsaturated mono- and dicarboxylic acids with amino alcohols which have a primary or secondary amino group, polyether acrylates and mixtures thereof.

5. A composition as claimed in claim 1, where the copolymer A1) comprises, in copolymerized form,
a) vinylimidazole,
b) N-vinylpyrrolidone,
c) at least one nonionic water-soluble monomer which is chosen from N-vinylamides of saturated C₁-C₈-monocarboxylic acids and primary amides of α,β-ethylenically unsaturated monocarboxylic acids and N-alkyl and N,N-dialkyl derivatives thereof which, in addition to the carbonyl carbon atom of the amide group, have at most 8 further carbon atoms,
d) at least one monomer which is chosen from acid salts and quaternization products of vinylimidazole, dimethylaminopropylmethacrylamide and the acid salt and quaternization products of dimethylaminopropylmethacrylamide.

6. A composition as claimed in claim 1, where the copolymer A1) comprises, in copolymerized form,
a) 0.5 to 40% by weight of vinylimidazole and/or a derivative thereof,
b) 20 to 99% by weight of at least one N-vinyllactam,
c) 0 to 50% by weight of at least one nonionic water-soluble monomer which is different from, and copolymerizable with, components a) and b), and
d) 0 to 30% by weight of at least one monomer which is chosen from α,β-ethylenically unsaturated water-soluble compounds with cationogenic and/or cationic hydrophilic groups.

7. A composition as claimed in claim 1, where the copolymer A1) comprises, in copolymerized form,
a) 1 to 20% by weight of vinylimidazole and/or a derivative thereof,
b) 20 to 80% by weight of at least one N-vinyllactam,
c) 5 to 50% by weight of at least one nonionic water-soluble monomer which is different from, and copolymerizable with, components a) and b), and
d) 0 to 30% by weight of at least one monomer which is chosen from α,β-ethylenically unsaturated water-soluble compounds with cationogenic and/or cationic hydrophilic groups.

8. A composition as claimed in claim 1, where the copolymer A1) comprises, in copolymerized form,
a) 1 to 10% by weight of vinylimidazole and/or a derivative thereof,
b) 30 to 70% by weight of at least one N-vinyllactam,
c) 10 to 40% by weight of at least one nonionic water-soluble monomer which is different from, and copolymerizable with, components a) and b), and
d) 1 to 20% by weight of at least one monomer which is chosen from α,β-ethylenically unsaturated water-soluble compounds with cationogenic and/or cationic hydrophilic groups.

9. A composition as claimed in claim 1, where component A2) comprises at least one acid-group-containing polymer which comprises, in copolymerized form, at least one monomer which contains a free-radically polymerizable, α,β-ethylenically unsaturated double bond and at least one anionogenic and/or anionic group per molecule.

10. A composition as claimed in claim 1, where component A2) comprises at least one carboxylic-acid-group-containing polyurethane.

11. A composition as claimed in claim 1, where component B) is chosen from
i) water,
ii) water-miscible organic solvents, preferably C₂-C₄-alkanols, in particular ethanol
iii) oils, fats, waxes,
iv) esters of C₆-C₃₀-monocarboxylic acids with mono-, di or trihydric alcohols which are different from iii),
v) saturated acyclic and cyclic hydrocarbons,
vi) fatty acids
vii) fatty alcohols
viii) propellant gases
and mixtures thereof.

12. A composition as claimed in claim 1 comprising at least one additive different from components A) and B) which is chosen from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tinting agents, tanning agents, dyes, pigments, consistency-imparting agents, humectants, refatting agents, collagen, protein hydrolyzates, lipids, antioxidants, antifoams, antistats, emollients and softeners.

13. A composition as claimed in claim 1 in the form of a gel, foam, spray, mousse, ointment, cream, emulsion, suspension, lotion, milk or paste.

14. A composition as claimed in claim 1 in the form of a spray, where the carboxylic-acid-group-containing polymer A2) comprises, in copolymerized form,
i) 60 to 90% by weight of at least one compound of the formula I 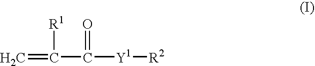
in which
R¹ is hydrogen or C₁-C₈-alkyl,
Y¹ is O, NH or NR³, and
R² and R³, independently of one another, are C₁-C₃₀-alkyl or C₅-C₈-cycloalkyl, where the alkyl groups may be interrupted by up to four nonadjacent heteroatoms or heteroatom-containing groups which are chosen from O, S and NH,
ii) 10 to 25% by weight of acrylic acid and/or methacrylic acid,
iii) 0 to 30% by weight of at least one monomer which is different from, and copolymerizable with, components i) and ii),
or where the carboxylic-acid-group-containing polymer A2) is a polyurethane.

15. A composition as claimed in claim 1 in the form of a mousse, where the carboxylic-acid-group-containing polymer A2) comprises, in copolymerized form,
i) 45 to 85% by weight of at least one compound of the formula I 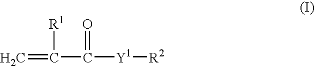
in which
R¹ is hydrogen or C₁-C₈-alkyl,
Y¹ is O, NH or NR³, and
R² and R³, independently of one another, are C—C₃₀-alkyl or C₅-C₈-cycloalkyl, where the alkyl groups may be interrupted by up to four nonadjacent heteroatoms or heteroatom-containing groups which are chosen from O, S and NH,
ii) 20 to 55% by weight of acrylic acid and/or methacrylic acid,
iii) 0 to 30% by weight of at least one monomer which is different from, and copolymerizable with, components i) and ii).

16. A composition as claimed in claim 1 in the form of a gel, where the carboxylic-acid-group-containing polymer A2) comprises, in copolymerized form,
i) 45 to 85% by weight of at least one compound of the formula I 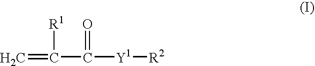
in which
R¹ is hydrogen or C₁-C₈-alkyl,
Y¹ is 0, NH or NR³, and
R² and R³, independently of one another, are C₁-C₃₀-alkyl or C₅-C₈-cycloalkyl, where the alkyl groups may be interrupted by up to four nonadjacent heteroatoms or heteroatom-containing groups which are chosen from O, S and NH,
ii) 20 to 60% by weight of acrylic acid and/or methacrylic acid,
iii) 5 to 50% by weight of at least one compound of the formula II 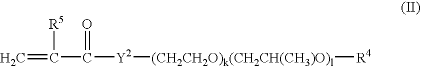
in which
the order of the alkylene oxide units is arbitrary,
k and l, independently of one another, are an integer from 0 to 1000, where the sum of k and l is at least 5,
R⁴ is hydrogen or C₁-C₃₀-alkyl,
R⁵ is hydrogen or C₁-C₈-alkyl,
Y² is O or NR⁶, where R⁶ is hydrogen, C₁-C₃₀-alkyl or C₅-C₈-cycloalkyl,
iv) 0 to 20% by weight of at least one monomer which is different from, and copolymerizable with, components i) to iii), and
v) 0.1 to 3% by weight of at least one crosslinking monomer with at least two ethylenically unsaturated, nonconjugated double bonds.

17. A composition as claimed in claim 1 in the form of a gel, where the carboxylic-acid-group-containing polymer A2) comprises, in copolymerized form,
i) 90 to 99.9% by weight of acrylic acid and/or methacrylic acid,
ii) 0 to 9.9% by weight of at least one monomer which is different from, and copolymerizable with, component i),
iii) 0.1 to 3% by weight of at least one crosslinking monomer with at least two ethylenically unsaturated, nonconjugated double bonds.

18. A composition as claimed in claim 1, which additionally comprises at least one nonionic thickener.

19. The use of a polyelectrolyte complex as defined in claim 1 in skin-cleansing compositions, compositions for the care and protection of the skin, nailcare compositions, preparations for decorative cosmetics and hair-treatment compositions.

20. The use as claimed in claim 19 in hair-treatment compositions as setting agents and/or as conditioners.

21. The use as claimed in claim 20, where the composition is in the form of a hair gel, shampoo, setting foam, hair tonic, hairspray or hair mousse.

22. The use of a polyelectrolyte complex as defined in claim 1 as auxiliary in pharmacy, preferably as or in (a) coating(s) for solid drug forms, for modifying rheological properties, as surface-active compound, as or in (an) adhesive(s), and as or in (a) coating(s) for the textile, paper, printing and leather industry.
